(19) United States
(12) Patent Application Publication
(10) Pub. No.: US 2024/0352400 A1
(43) Pub. Date: Oct. 24, 2024

(54) GROWTH MEDIA COMPOSITIONS AND GROWTH ENVIRONMENTAL CONDITIONS FOR IMPROVED AERIAL MYCELIUM

(71) Applicant: Ecovative Design LLC, Green Island, NY (US)

(72) Inventors: Jacob Michael Winiski, Troy, NY (US); Karl John Kiser, Cambridge, NY (US)

(21) Appl. No.: 18/642,656

(22) Filed: Apr. 22, 2024

Related U.S. Application Data
(60) Provisional application No. 63/498,003, filed on Apr. 24, 2023.
(60) Provisional application No. 63/505,675, filed on Jun. 1, 2023.
(60) Provisional application No. 63/516,425, filed on Jul. 28, 2023.

(51) Int. Cl. C12N 1/14

(52) U.S. Cl. CPC C12N 1/14; C12N 2500/02; C12N 2500/34; C12N 2500/36; C12N 2501/90; C12N 2501/998

(57) ABSTRACT

Methods of cultivation of aerial mycelium (from extra-particle aerial mycelial growth), with improved growth medium compositions and environmental conditions are described herein. The methods and growth medium compositions provide a scalable method of culturing extra-particle aerial mycelium, and ultimately aerial mycelium, that satisfies a plurality of material properties for a variety of mycelium-based end products.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### CROSS-REFERENCE TO RELATED APPLICATIONS

This application is related to U.S. Provisional Patent Application No. 63/498,003, filed Apr. 24, 2023, entitled “GROWTH MEDIA COMPOSITIONS AND GROWTH ENVIRONMENTAL CONDITIONS FOR IMPROVED AERIAL MYCELIUM”; U.S.

Provisional Patent Application No. 63/505,675, filed Jun. 1, 2023, entitled “GROWTH MEDIA COMPOSITIONS AND GROWTH ENVIRONMENTAL CONDITIONS FOR IMPROVED AERIAL MYCELIUM”; U.S. Provisional Patent Application No. 63/516,425, filed Jul. 28, 2023, entitled “GROWTH MEDIA COMPOSITIONS AND GROWTH ENVIRONMENTAL CONDITIONS FOR IMPROVED AERIAL MYCELIUM,” the disclosures of each which is incorporated herein by reference in its entirety.

### BACKGROUND

### Field

This application relates generally to improved methods and systems of growing aerial mycelium, and in particular, for growth medium compositions and growth environmental conditions for improved aerial mycelium.

### Background

The present application relates to improved systems, apparatus, and methods of growing an aerial mycelium material, and in particular, for growing aerial mycelium. The aerial mycelium may be used as a food or a textile product or ingredient(s) thereof. Such a food product or ingredient can include edible aerial mycelium having a texture, e.g., that is analogous to a muscle meat product, such as, e.g., mycelium-based bacon, or other animal-based meat alternatives. Such a textile product or ingredient can be used in the manufacture of mycelium-based textile products, leather-like or petroleum-based material alternatives, composites, or foams.

### SUMMARY

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, a mycelium growth medium composition includes a nutrition profile configured to grow an aerial mycelium with pre-determined material properties and includes a protein content of at least 10% (w/w); a non-fibrous carbohydrate content of at least 30% by (w/w); a total starch content of less than 5% by (w/w); an ethanol-soluble carbohydrate content of at least 4% (w/w); a fat content of less than 5% (w/w); and a carbon-to-nitrogen ratio at least 35 but less than 50.

In some aspects, the mycelium growth medium composition includes a protein content of at least 10% (w/w) but less than 11% (w/w).

In some aspects, the mycelium growth medium composition includes a non-fibrous carbohydrate content of greater than 30% (w/w) but less than 32% (w/w).

In some aspects, the mycelium growth medium composition includes a total starch content of at least 4% (w/w) but less than 5% (w/w).

In some aspects, the mycelium growth medium composition includes an ethanol-soluble carbohydrate content of at least 4% (w/w) but less than 6% (w/w).

In some aspects, the mycelium growth medium composition of claim includes a fat content of at least 4% (w/w) but less than 5% (w/w).

In some aspects, the mycelium growth medium composition includes a carbon-to-nitrogen ratio of at least 35 but less than 40.

In another aspect, a method of culturing an aerial mycelium includes preparing a mycelium growth medium with a nutrition profile including: a protein content of at least 10% (w/w); a non-fibrous carbohydrate content of at least 30% (w/w); a total starch content of less than 5% (w/w); an ethanol-soluble carbohydrate content of at least 4% (w/w); a fat content of less than 5% (w/w); and a carbon-to-nitrogen ratio at least 35 but less than 50; hydrating the growth medium; sterilizing the growth medium; inoculating the growth medium with a fungal inoculum; and placing the inoculated growth medium into a growth environment configured to control a plurality of environmental conditions.

In some aspects, the method of culturing an aerial mycelium includes a plurality of environmental conditions, which include: a temperature, a CO₂ content, an airflow velocity, a mist deposition rate, and a mist composition.

In some aspects, the method of culturing an aerial mycelium includes a CO₂ content of at least 3.5% by volume of the growth environment.

In some aspects, the method of culturing an aerial mycelium includes a CO₂ content varies 0.25% over an incubation time period.

In some aspects, the method of culturing an aerial mycelium includes the CO₂ content increasing during a first period of an incubation time period at a rate of at least 0.4% per hour to a target CO₂ level, and including the CO₂ content being maintained at the target CO₂ level during a second period of the incubation time period.

In some aspects, the method of culturing an aerial mycelium includes aCO₂ content reaching 50% of the target CO₂ level within twenty hours of the beginning of the incubation time period.

In some aspects, the method of culturing an aerial mycelium includes an airflow velocity of between 30 ft/min and 60 ft/min.

In some aspects, the method of culturing an aerial mycelium includes a mist deposition rate of between 0.1 mg/cm²/hour and 0.6 mg/cm²/hour.

In some aspects, the method of culturing an aerial mycelium includes a mist composition including tap water with a minimum conductivity of at least 100 μS/cm.

In some aspects, the method of culturing an aerial mycelium includes a fungal inoculum being a Ganoderma sessile inoculum.

In some aspects, the techniques described herein relate to a mycelium growth medium composition with a nutritional profile configured to grow an aerial mycelium with pre-determined material properties, the nutrition profile including: a protein content of at least 10% (w/w); a non-fibrous carbohydrate content of less than 25% (w/w); a total starch content of less than 4% (w/w); an ethanol-soluble carbohydrate content of less than 2.5% (w/w); a fat content of less than 2% (w/w); and a carbon-to-nitrogen ratio of at least 25 but less than 35.

In another aspect, a mycelium growth medium composition with a nutrition profile configured to grow an aerial mycelium with pre-determined material properties, the nutrition profile including: a protein content of at least 9% (w/w); a non-fibrous carbohydrate content of at least 30% (w/w); a total starch content of less than 5% (w/w); an ethanol-soluble carbohydrate content of at least 2.5% (w/w); a fat content of less than 2% (w/w); and a carbon-to-nitrogen ratio at least 25 but less than 40, including the modified nutrition profile being configured to support an aerial mycelium growth during an aerial growth phase.

In some aspects, the mycelium growth medium composition including the nutrition profile includes one or more growth medium components.

In some aspects, the mycelium growth medium composition including the one or more growth medium components includes at least one simple sugar.

In some aspects, the mycelium growth medium composition including the at least one simple sugar, includes at least one of arabinose, cellobiose, dextrin, dextrose, fructose, fucose, galactose, gentiobiose, glucosamine, glucose, lactose, lactulose, maltodextrin, maltose, maltotriose, mannose, melezitose, melibiose, sucrose, trehalose, xylose, or a combination thereof.

In some aspects, the mycelium growth medium composition including the one or more growth medium components further includes at least one of a protein source, a fat source, a fiber source, a non-fiber carbohydrate source, a starch carbohydrate source, an ethanol-soluble carbohydrate source, or an ash content.

In some aspects, the mycelium growth medium composition including the nutrition profile further includes a remainder portion, the remainder portion including at least one of: a fiber content and an ash content.

In some aspects, a mycelium growth matrix includes the mycelium growth medium and a fungus.

In some aspect, a system for growing an aerial mycelium includes: a pre-colonization receptacle containing the mycelium growth matrix; an aerial growth tool; and an aerial growth phase growth matrix supported by the aerial growth tool, the aerial growth phase growth matrix having a nutrition profile configured to grow an aerial mycelium with pre-determined material properties, the nutrition profile including: a protein content of at least 9% (w/w); a non-fibrous carbohydrate content of at least 30% (w/w); a total starch content of less than 5% (w/w); an ethanol-soluble carbohydrate content of at least 2.5% (w/w); a fat content of less than 2% (w/w); and a carbon-to-nitrogen ratio at least 25 but less than 40, including the modified nutrition profile being configured to support an aerial mycelium growth during an aerial growth phase.

In another aspect, a method of culturing an aerial mycelium includes preparing a pre-colonization phase growth medium with a first nutrition profile; inoculating the pre-colonization growth medium with a fungal inoculum to create a pre-colonization growth matrix; incubating the pre-colonization growth matrix for a pre-colonization period; modifying the first nutrition profile of the pre-colonization growth matrix to create an aerial growth phase growth matrix with a second nutrition profile; applying a casing layer to the aerial growth phase growth matrix; and placing the aerial growth phase growth matrix into a growth environment configured to control a plurality of environmental conditions.

In some aspects, a method of culturing an aerial mycelium includes a first nutrition profile including: a protein content of at least 10% (w/w); a non-fibrous carbohydrate content of less than 25% (w/w); a total starch content of less than 4% (w/w); an ethanol-soluble carbohydrate content of less than 2.5% (w/w); a fat content of less than 2% (w/w); and a carbon-to-nitrogen ratio at least 25 but less than 35.

In some aspects, a method of culturing an aerial mycelium includes modifying the first nutrition profile including adding a simple sugar component to create an aerial growth phase growth matrix with a second nutrition profile.

In some aspects, a method of culturing an aerial mycelium including the second nutrition profile including: a protein content of at least 9% (w/w); a non-fibrous carbohydrate content of at least 30% (w/w); a total starch content of less than 5% (w/w); an ethanol-soluble carbohydrate content of at least 2.5% (w/w); a fat content of less than 2% (w/w); and a carbon-to-nitrogen ratio at least 25 but less than 40.

In some aspects, a method of culturing an aerial mycelium includes the casing layer including an aerial growth phase growth medium including the second nutrition profile.

In some aspects, a method of culturing an aerial mycelium includes the casing layer being between 10% (w/w) and 25% (w/w) of the aerial growth phase growth matrix.

In some aspects, a method of culturing an aerial mycelium includes a thickness of the casing layer being between 0.25 cm and 2 cm.

In some aspects, a method of culturing an aerial mycelium includes the fungal inoculum being a Ganoderma sessile inoculum.

In some aspects, a method of culturing an aerial mycelium includes inoculating the pre-colonization phase growth medium including inoculating the pre-colonization phase growth medium at an inoculation rate.

In some aspects, a method of culturing an aerial mycelium includes an inoculation rate being 15% (wet mass spawn/dry mass aerial growth phase growth medium).

In some aspects, a method of culturing an aerial mycelium includes a plurality of environmental conditions including: a temperature, a CO₂ content, an airflow velocity, a mist deposition rate, a mist composition, and a mist duty cycle.

In some aspects, a method of culturing an aerial mycelium includes a CO₂ content being at least 2.34% by volume of the growth environment.

In some aspects, a method of culturing an aerial mycelium includes a CO₂ content varying less than 1.5% over an incubation time period.

In some aspects, a method of culturing an aerial mycelium includes a CO₂ content increasing during a first period of an incubation time period at a rate of at least 0.4% per hour to a CO₂ level, and including the CO₂ content being maintained at the CO₂ level during a second period of the incubation time period.

In some aspects, a method of culturing an aerial mycelium includes a CO₂ content reaching 50% of the CO₂ level within twenty hours of the beginning of the incubation time period.

In some aspects, a method of culturing an aerial mycelium includes an airflow velocity being between 40 ft/min and 60 ft/min.

In some aspects, a method of culturing an aerial mycelium includes a mist deposition rate of between 0.1 mg/cm²/hour and 0.5 mg/cm²/hour.

In some aspects, a method of culturing an aerial mycelium includes a mist composition including tap water with a minimum conductivity of at least 100 μS/cm.

In some aspects, a method of culturing an aerial mycelium includes a mist duty cycle including a duty cycle of at least 75% to 100% and a duty cycle period of about 10 seconds to about 60 seconds.

In another aspect, a method of culturing an aerial mycelium includes inoculating a growth medium with a fungal inoculum to create a growth matrix; and placing the growth matrix into a growth environment without a casing layer, the growth environment configured to control a plurality of environmental conditions including a CO₂ variance, including the CO₂ variance and the lack of the casing layer being configured to produce the aerial mycelium with a desired morphology.

In another aspect, a method of culturing an aerial mycelium includes inoculating a growth medium with a fungal inoculum to create a growth matrix; applying a casing layer to the growth matrix; and placing the growth matrix with the casing layer into a growth environment, the growth environment configured to control a plurality of environmental conditions including a CO₂ variance, including the CO₂ variance and the casing layer being configured to produce the aerial mycelium with a desired morphology.

In some aspects, a method of culturing an aerial mycelium includes preparing a pre-colonization phase growth medium with a first nutrition profile, the first nutrition profile including a pre-colonization phase crude protein content and a pre-colonization phase non-fiber carbohydrate content; inoculating the pre-colonization growth medium with a fungal inoculum to create a pre-colonization growth matrix; incubating the pre-colonization growth matrix for a pre-colonization period; adding a simple sugar component to modify the non-fiber carbohydrate content of the first nutrition profile of the pre-colonization growth matrix to create an aerial growth phase growth matrix with a second nutrition profile, including the second nutrition profile including an aerial growth phase crude protein content and an aerial growth phase non-fiber carbohydrate content; and placing the aerial growth phase growth matrix into a growth environment configured to control a plurality of environmental conditions, including a length of the pre-colonization period and the modification of the first nutrition profile to create the second nutrition profile being configured to produce the aerial mycelium with a desired morphology and a desired bioefficiency.

In some aspects, the method of culturing an aerial mycelium includes a pre-colonization phase crude protein content being greater than 10% (w/w) and less than about 12% (w/w), and including a simple sugar component modifying the first nutrition profile such that the aerial growth phase non-fiber carbohydrate content is greater than 3% (w/w) but less than 3.5% (w/w).

In some aspects, the method of culturing an aerial mycelium includes the pre-colonization phase crude protein content being greater than 8% (w/w) but less than 10% (w/w), and including a simple sugar component modifying the first nutrition profile such that the aerial growth phase non-fiber carbohydrate content is greater than 4% (w/w) but less than 4.5% (w/w).

In another aspect, the techniques described herein relate to all systems, devices, and methods disclosed herein.

In another aspect, a method of culturing an aerial mycelium includes preparing a growth medium with a moisture content; inoculating the growth medium with a fungal inoculum to create a growth matrix; incubating the growth matrix for a pre-colonization period; and placing the growth matrix into a growth environment for an aerial growth phase.

In some aspects, a method of culturing an aerial mycelium includes a moisture content being about 70%, and a pre-colonization period being three days or less.

In some aspects, a method of culturing an aerial mycelium includes a moisture content being at least 55% and less than 65%, and a pre-colonization period being four days or more.

In some aspects, a method of culturing an aerial mycelium includes a moisture content being about 63%.

In another aspect, a method of culturing an aerial mycelium includes preparing a pre-colonization phase growth medium with a moisture content; inoculating the pre-colonization growth medium with a fungal inoculum to create a pre-colonization growth matrix; incubating the pre-colonization growth matrix for a pre-colonization period; modifying the pre-colonization growth matrix to create an aerial growth phase growth matrix; and placing the aerial growth phase growth matrix into a growth environment configured to control a plurality of environmental conditions.

In some aspects, the method of culturing an aerial mycelium includes a moisture content being about 70%, and the pre-colonization period being three days or less.

In some aspects, the method of culturing an aerial mycelium includes a moisture content being at least 55% and less than 65%.

In some aspects, the method of culturing an aerial mycelium includes a moisture content being about 63%.

In some aspects, the method of culturing an aerial mycelium includes a pre-colonization period being four days or more.

In some aspects, the method of culturing an aerial mycelium further includes applying a casing layer to the aerial growth phase growth matrix.

Alternative or additional embodiments described herein provide a method of culturing an aerial mycelium comprising one or more of the features of the foregoing description or of any description elsewhere herein.

Alternative or additional embodiments described herein provide a system for growing an aerial mycelium comprising one or more of the features of the foregoing description or of any description elsewhere herein.

Alternative or additional embodiments described herein provide a mycelium growth medium composition comprising one or more of the features of the foregoing description or of any description elsewhere herein.

### BRIEF DESCRIPTION OF THE DRAWINGS

The features and advantages of the methods and compositions described herein will become more fully apparent from the following description and appended claims, taken in conjunction with the accompanying drawings. These drawings depict only several embodiments in accordance with the disclosure and are not to be considered limiting of their scope. In the drawings, similar reference numbers or symbols typically identify similar components, unless context dictates otherwise. In some instances, the drawings may not be drawn to scale.

FIG. 1A illustrates an embodiment of a growth matrix suitable to support extra-particle aerial mycelial growth.

FIG. 1B illustrates an embodiment of extra-particle aerial mycelial growth extending from the growth matrix of FIG. 1A.

FIGS. 2A-D are illustrative plots showing the predicted improved CO₂ parametrization against the full experimental distribution evaluated.

FIGS. 3A-E are illustrative plots showing the predicted optimum mist deposition parametrization against the full experimental distribution evaluated.

FIGS. 4A-F are illustrative plots showing the predicted optimum substrate composition with respect to individual components thereof.

FIGS. 5A-C and 6A-C show the ranked effects between environmental and substrate nutrition parameters.

FIGS. 7A-C depict cultivation paradigm model improvements for three experiments.

FIG. 8 shows a flow diagram describing the process of cultivating extra-particle aerial mycelial growth, ultimately producing aerial mycelium.

FIG. 9-12 illustrates visual colonization of pre-colonized substrate at 72-hours and 96-hours from incubation.

FIGS. 13A-B provide target and threshold values for environmental parameters.

FIGS. 14A-C and 15A-C show ranked effects between growth qualities and environmental parameters.

FIG. 16-17 provides target and threshold values for substrate incubation, casing layer, packing density and composition parameters.

FIGS. 18A-C show ranked effects between defined substrate composition and growth qualities.

FIG. 19 provides the aerial mycelium growth qualities resulting from operationalizing the aerial mycelium cultivation parameters.

FIGS. 20A-C illustrate data showing morphological principal components and morphological segments comparing the full experimental population based on whether an uninoculated casing layer was or was not applied.

FIGS. 21A-C illustrate data with significant interactive relationships between variance in environmental CO₂ and application of an uninoculated casing layer.

FIG. 22 illustrates the relationship between morphological Principle Component 1 (PC1) and mean chamber airflow velocity.

FIG. 23 illustrates data showing a second-order fit of dry mass (DM) bioefficiency as a combined function of precolonization and substrate C/N ratio.

FIGS. 24A-C illustrate data demonstrating the effect of modifying the nutritional composition of growth media.

FIGS. 25A-C illustrate relationships between crude protein and pre-colonization.

FIG. 26 illustrates data showing the relationship of bioefficiency as a combined function of crude protein and ethanol soluble carbohydrates.

FIG. 27 illustrates data of the relationship between bioefficiency and pre-colonization days.

### DETAILED DESCRIPTION

U.S. Pat. No. 11,277,979 to Greetham et al., International PCT Patent Application No. WO2019/099474A1 to Kaplan-Bie et al., PCT Patent Application No. WO2023172696 to Snyder et al., PCT Patent Application No. WO2022235688 to Winiski et al., and PCT Patent Application No. WO2022235694 to Carlton et al., the entirety of each of which is incorporated herein by reference thereto except where inconsistent with the disclosure herein. The methods of the current disclosure utilize environmental conditions as described in the preceding references to provide more consistently repeatable growth systems that are adaptable to changing environmental conditions, to accommodate the needs of varying fungal organisms, to different growth equipment paradigms, and to target differing attributes desired in various mycelium products. Still further, the disclosed systems offer the potential for energy and resource efficiency as a result of closely monitored conditions and thoughtful action steps taken in relation thereto, (after considering multiple implications and variables) while still providing for high quality and quantity mycological (e.g., mycelium-based) material.

### Definitions

“Mycelium” as used herein refers to a connective network of fungal hyphae, with mycelia being the plural form of mycelium.

“Hyphae” as used herein refers to branched filament vegetative cellular structures that are interwoven to form mycelium.

“Substrate” as used herein refers to a material or surface thereof, from or on which an organism lives, grows, and/or obtains its nourishment. In some embodiments, a substrate provides sufficient nutrition to the organism under target growth conditions such that the organism can live and grow without providing the organism a further source of nutrients. A variety of substrates are suitable to support the growth of an extra-particle aerial mycelium, and ultimately aerial mycelium of the present disclosure. Suitable substrates are disclosed, for example, in US20200239830A1 to O'Bren et al., the entire contents of which are hereby incorporated by reference in their entirety to the extent not inconsistent with the content of this disclosure. In some embodiments, the substrate is a natural substrate. Non-limiting examples of a natural substrate include a lignocellulosic substrate, a cellulosic substrate, or a lignin-free substrate. A natural substrate can be an agricultural waste product or one that is purposefully harvested for the intended purpose of food production, including mycelial-based food production. Further non-limiting examples of substrates suitable for supporting the growth of mycelia of the present disclosure include soy-based materials, oak-based materials, maple-based materials, corn-based materials, seed-based materials and the like, or combinations thereof. The materials can have a variety of particle sizes, as disclosed in US2020/0239830A1 to O'Brien et al., and occur in a variety of forms, including shavings, pellets, chips, flakes, or flour, or can be in monolithic form. Non-limiting examples of suitable substrates for the production of mycelia of the present disclosure include corn stover, maple flour, maple flake, maple chips, soy flour, chickpea flour, millet seed flour, oak pellets, soybean hull pellets, various seeds, and combinations thereof. Additional useful substrates for the growth of mycelia are disclosed herein.

“Growth medium” or “growth media” as used herein refers to a matrix containing a substrate and an optional further source of nutrition that is the same or different than the substrate, wherein the substrate, the nutrition source, or both are intended for fungal consumption to support mycelial growth. In some embodiments, the further sources of nutrition may include simple sugars, nutritional supplements, etc. Each component of the growth medium, including the substrate, may have a component nutrition profile, which may be combined into a composite growth medium nutrition profile. Combined, each of these growth medium components/constituents can define a nutrition profile of the growth medium.

“Growth matrix” as used herein refers to a matrix containing a growth medium and a fungus. In some embodiments, the fungus is provided as a fungal inoculum; thus, in such embodiments, the growth matrix comprises a fungal-inoculated growth medium. In other embodiments, the growth matrix comprises a colonized substrate.

“Inoculated substrate” as used herein refers to a substrate that has been inoculated with fungal inoculum. For example, an inoculated substrate can be formed by combining an uninoculated substrate with a fungal inoculum. An inoculated substrate can be formed by combining an uninoculated substrate with a previously inoculated substrate. An inoculated substrate can be formed by combining an inoculated substrate with a colonized substrate.

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth medium substrate. The colonized substrate may further contain residual nutrition that has not been consumed by the colonizing fungus. As is understood by persons of ordinary skill in the art, a colonized substrate has undergone primary myceliation, sometimes referred to by skilled artisans as having undergone a “mycelium run.” Thus, in some particular aspects, a colonized substrate consists essentially of a substrate and a colonizing fungus in a primary myceliation phase. For many fungal species, asexual sporulation occurs as part of normal vegetative growth, and as such could occur during the colonization process. Accordingly, in some embodiments, a colonized substrate of the present disclosure may also contain asexual spores (conidia). In some aspects, a colonized substrate of the present disclosure can exclude growth progression into sexual reproduction and/or vegetative foraging. Sexual reproduction includes fruiting body formation (e.g., primordiation and differentiation) and sexual sporulation (meiotic sporulation). Vegetative foraging includes any mycelial growth away from the colonizing substrate (such as aerial growth). Thus, in some further aspects, a colonized substrate can exclude mycelium that is in a vertical expansion phase of growth. A colonized substrate can enter a mycelial vertical expansion phase during incubation in a growth environment of the present disclosure. For example, a colonized substrate can enter a mycelial vertical expansion phase upon introducing mist into the growth environment and/or depositing mist onto colonized substrate and/or any ensuing extra-particle growth. In some embodiments, the use of mist can be adjusted, for example, to desired levels and timing, to affect the topology, morphology, density, and/or volume of the growth.

Any suitable substrate can be used alone, or optionally combined with a nutrient source, as medium to support mycelial growth. The growth medium can be hydrated to a final target moisture content prior to inoculation with a fungal inoculum. In a non-limiting example, the substrate or growth medium can be hydrated to a final moisture content of at least about 50% (w/w), at most about 95% w/w, within a range of about 50% to about 95%, about 50% to about 90%, about 50% to 85%, about 50% (w/w) to about 80% (w/w), about 50% (w/w) to about 75% (w/w), within a range of about 50% (w/w) to about 65% (w/w), within a range of about 50% (w/w) to about 60% (w/w), or within a range of about 60% (w/w) to about 70% (w/w). Growth medium hydration can be achieved via the addition of any suitable source of moisture. In a non-limiting example, the moisture source can be airborne or non-airborne liquid phase water (or other liquids), an solution containing one or more additives (including but not limited to a nutrient source), and/or gas phase water (or other compound). In some embodiments, at least a portion of the moisture is derived from steam utilized during bioburden reduction of the growth medium. In some embodiments, inoculation of the growth medium with the fungal inoculum can include a further hydration step to achieve a target moisture content, which can be the same or different than the moisture content of the growth medium. For example, if growth medium loses moisture during fungal inoculation, the fungal inoculated growth medium can be hydrated to compensate for the lost moisture.

Methods for the production of extra-particle aerial mycelial growth, and ultimately aerial mycelium, disclosed herein can include an inoculation stage, wherein an inoculum is used to transport an organism into a substrate. The inoculum, which carries a desired fungal strain, is produced in sufficient quantities to inoculate a target quantity of substrate. The inoculation can provide a plurality of myceliation sites (nucleation points) distributed throughout the substrate. Inoculum can take the form of a liquid, a slurry, or a solid, or any other known vehicle for transporting an organism from one growth-supporting environment to another. Generally, the inoculum comprises water, carbohydrates, sugars, vitamins, other nutrients, and fungi. The inoculum may contain enzymatically available carbon and nitrogen sources (e.g., lignocellulosic biomass, chitinous biomass, carbohydrates) augmented with additional micronutrients (e.g., vitamins, minerals). The inoculum can contain inert materials (e.g., perlite). In a non-limiting example, the fungal inoculum can be a seed-supported fungal inoculum, a feed-grain-supported fungal inoculum, a seed-sawdust mixture fungal inoculum, or another commercially available fungal inoculum, including specialty proprietary types provided by inoculum retailers. In some aspects, a fungal inoculum can be characterized by its density. In some embodiments, a fungal inoculum has a density of about 0.1 gram per cubic inch to about 10 grams per cubic inch, or from about 1 gram per cubic inch to about 7 grams per cubic inch. A skilled person can modify variables including the substrate or growth medium component identities, substrate or growth medium nutrition profile, substrate or growth medium moisture content, substrate or growth medium bioburden, inoculation rate, and inoculum constituent concentrations to arrive at a suitable medium to support aerial mycelial growth. In some embodiments, the inoculation rate can be expressed as a percentage of the target volume of the substrate or growth medium (% (v/v)). In some embodiments, the inoculation rate can range from about 0.1% (v/v) to about 80% (v/v). In some embodiments, the inoculation rate is at most about 50% (v/v), at most about 45% (v/v), at most about 40% (v/v), at most about 30% (v/v), at most about 25% (v/v), at most about 20% (v/v), at most about 15% (v/v), at most about 10% (v/v) or at most about 5% (v/v). In some embodiments, the inoculation rate is about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 11% (v/v), about 12% (v/v), about 13% (v/v), about 14% (v/v), about 15% (v/v), about 16% (v/v), about 17% (v/v), about 18% (v/v), about 19% (v/v), about 20% (v/v), about 21% (v/v), about 22% (v/v), about 23% (v/v), about 24% (v/v), about 25% (v/v), about 26% (v/v), about 27% (v/v), about 28% (v/v), about 29% (v/v) or about 30% (v/v); or any range therebetween. In some embodiments, the inoculation rate can be expressed as a percentage of the target dry mass of the substrate or growth medium (% (w/w)). In some embodiments, the inoculation rate can range from about 0.1% (w/w) to about 80% (w/w). In some embodiments, the inoculation rate is at most about 50% (w/w), at most about 45% (w/w), at most about 40% (w/w), at most about 30% (w/w), at most about 25% (w/w), at most about 20% (w/w), at most about 15% (w/w), at most about 10% (w/w) or at most about 5% (w/w). In some embodiments, the inoculation rate is about 1% (w/w), about 2% (w/w), about 3% (w/), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w) or about 30% (w/w); or any range therebetween.

“Aerial mycelium” as used herein refers to mycelium obtained from extra-particle aerial mycelial growth, and which is substantially free of growth matrix.

“Extra-particle aerial mycelium” as used herein refers to mycelium whose aerial mycelial growth occurs away from and outward from the surface of a growth matrix (hence “extra-particle”). In some embodiments, the extra-particle aerial mycelium includes aerial hyphae of a mycelium growing in and/or on a colonized substrate. For example, a growing mycelium in and/or on a colonized substrate can produce hyphae that emerge from and proliferate independently of direct contact with the growth substrate, thereby producing a portion of the mycelium that does not include substrate or growth matrix. In some embodiments, extra-particle aerial mycelium can exhibit negative gravitropism. In a geometrically unrestricted scenario, extra-particle aerial mycelium growth can be positively, negatively or neutrally gravitropic. In some embodiments, extra-particle aerial mycelium growth can be radial, wherein hyphal growth expands in all directions from a point of inoculation and/or germination of, e.g., a growth matrix. In some embodiments, external inputs, such as airflow, can be applied to the extra-particle aerial mycelium as it grows, thereby affecting the direction of growth of hyphae. For example, downward airflow can be applied to extra-particle aerial mycelium growth in the direction of gravity. Alternatively, airflow can be applied across the growth matrix in a manner parallel or horizontal to the growth matrix surface (which contains substrate).

“Extra-particle aerial mycelial growth”, as used herein refers to a distinct mycelial growth that occurs away from and outward from the surface of a growth matrix (growth media/substrate), and which can exhibit negative gravitropism. The extra-particle aerial mycelial growth is associated with the growth of extra-particle aerial mycelium. In certain contexts throughout, the terms “extra-particle aerial mycelial growth” may refer to the noun extra-particle aerial mycelium, or the growth activity, depending on the context. In a geometrically unrestricted scenario, extra-particle aerial mycelial growth could be described as being negatively gravitropic, positively gravitropic, or neutrally gravitropic, aerial, and radial in which growth will expand in all directions from its point source. In some embodiments, external forces, such as airflow, can be applied towards (e.g., approximately perpendicular to) the growth substrate, and in some embodiments, through the growth substrate, for example, to create downward aerial mycelium growth in the direction of gravity. Alternatively, airflow can be applied across the growth substrate in a manner parallel to the growth substrate surface.

“Positive gravitropism” as used herein refers to growth that preferentially occurs in the direction of gravity.

“Negative gravitropism” as used herein refers to mycelial growth that preferentially occurs in the direction away from gravity. As disclosed herein, extra-particle aerial mycelial growth can exhibit negative gravitropism. Without being bound by any particular theory, this may be attributable at least in part to the geometric restriction of the growth format, wherein an uncovered tool (e.g., growth matrix or growth media support structure) having a bottom and side walls contains a growth matrix. With such geometric restriction, growth will primarily occur along the unrestricted dimension(s), which in the scenario is primarily vertically (negatively gravitropic) if the tool is positioned such that its opening is facing vertically upward in orientation.

Aerial mycelia of the present disclosure can be grown in a matter of weeks or days. This feature is of practical value in the production of food ingredients or food products, where time and efficiency are at a premium. Accordingly, the presently disclosed method of making an aerial mycelium comprises incubating a growth matrix in a growth environment for an incubation time period of up to about 3 weeks. In some embodiments, the incubation time period can be within a range of about 4 days to about 17 days. In some further embodiments, the incubation time period can be within a range of about 7 days to about 16 days, within a range of about 8 days to about 15 days, within a range of about 9 days to about 15 days, within a range of about 9 days to about 14 days, within a range of about 8 to about 14 days, within a range of about 7 days to about 13 days, or within a range of about 7 days to about 10 days. In some more particular embodiments, the incubation time period can be about 7 days, about 8 days, about 9 days, about 10 days, about 11 days, about 12 days, about 13 days, about 14 days, about 15 days or about 16 days, or any range therebetween.

“***”, “**”, “*” and “.” as used herein refers to levels of statistical significance of statistical analyses and/or tests contained herein. As understood by the skilled person in the art, these symbols indicate how statistically significant the results of a statistical analysis and/or test are in reference to a p-value. The p-value measures the probability of obtaining an effect at least as extreme as the one in the sample data, assuming the null hypothesis is true. A smaller p-value suggests that there is strong evidence in favor of the alternative hypothesis (i.e., non-null hypothesis). As used herein, “***” indicates a very high level of statistical significance, corresponding to a p-value of less than 0.001, suggesting that the probability of the observed effect occurring by chance is less than 0.1%. As used herein, “**” indicates a high level of statistical significance, corresponding to a p-value of less than 0.01, suggesting that the probability of the observed effect occurring by chance is less than 1.0%. As used herein, “*” indicates a moderate level of statistical significance, corresponding to a p-value of less than 0.05, suggesting that the probability of the observed effect occurring by chance is less than 5.0%. As used herein, “.” indicates a marginal level of statistical significance, corresponding to a p-value of less than 0.1, suggesting that the probability of the observed effect occurring by chance is less than 10%. As used herein, when no symbol is present for a statistical analysis and/or test, e.g. “ ”, this indicates little statistical significance, corresponding to a p-value of greater than 0.1, suggesting that the probability of the observed effect occurring by chance is greater than 10%.

Advantageously, incubating a growth matrix comprising a colonized substrate (wherein said colonized substrate comprises a growth medium pre-colonized with mycelium of a fungus) in a growth environment of the present disclosure can result in earlier expression of aerial mycelial tissue compared to incubation of a growth matrix comprising substantially the same or a similar growth medium and a fungal inoculum, wherein the fungal inoculum contains a fungus. Accordingly, a method of making an aerial mycelium of the present disclosure can comprise incubating a growth matrix comprising a colonized substrate (wherein said colonized substrate comprises a growth medium pre-colonized with mycelium of a fungus) in a growth environment for an incubation time period, and producing extra-particle aerial mycelial growth therefrom, wherein the incubation time period is at least about 1 day, at least about 2 days, at least about 3 days, or at least about 4 days less than the incubation time period for producing extra-particle aerial mycelial growth from a growth matrix comprising a growth medium and a fungal inoculum, wherein the fungal inoculum comprises a fungus.

In some other embodiments, the incubation time period ends no later than when a visible fruiting body forms. In a non-limiting example, the incubation time period can end prior to a karyogamy or meiosis phase of the fungal reproductive cycle. In some other embodiments, the incubation time period ends when a visible fruiting body forms. As disclosed herein, the extra-particle aerial mycelia, and ultimately the aerial mycelia, of the present disclosure can be prepared without the formation of a visible fruiting body, thus, in some embodiments, an incubation time period can end without regard to the formation of a visible fruiting body. Trial incubation runs can be used to inform the period of time in the growth environment during which sufficient extra-particle aerial mycelial growth product occurs (e.g., aerial mycelial growth of a predetermined thickness) without the formation of visible fruiting bodies.

### Definitions and Methods Related to Growth Environment

US Published Patent Application 2015/0033620 to Greetham et al., the entire contents of which are hereby incorporated by reference in their entirety to the extent not inconsistent with the content of this disclosure, describes techniques for growing a material comprising aerial mycelium. As described therein, a mycological biopolymer product provided by the disclosed method is characterized as containing a homogenous biopolymer matrix that is comprised predominantly of fungal chitin and trace residues (e.g., beta-glucan, proteins). The mycological biopolymer is up-cycled from domestic agricultural lignocellulosic waste and is made by inoculating the substrate made of domestic agricultural lignocellulosic waste with a selected fungus in a container that is sealed off from the ambient environment external to the container. In addition to the substrate and fungal inoculum, the container contains a void space. A network of undifferentiated aerial mycelium comprising a chitin-polymer grows into and fills the void space of the container. The chitin-polymer-based aerial mycelium is subsequently extracted from the substrate and dried. As further described in US 2015/0033620 to Greetham et al., the environmental conditions for producing the mycological biopolymer product described therein, i.e., a high carbon dioxide (CO₂) content (about 3% to about 7% by volume) and an elevated temperature (from about 85° F. to about 95° F.), prevent full differentiation of the fungus into a mushroom, as evidenced by the absence of a visible fruiting body.

In one embodiment, the present disclosure provides an aerial mycelium. In a further embodiment, the aerial mycelium does not contain a visible fruiting body.

As described in WO 2019/099474A1 to Kaplan-Bie et al., the entire contents of which are hereby incorporated by reference in their entirety to the extent not inconsistent with the content of this disclosure, another method of growing a mycological biopolymer material employs incubation of a substrate with nutritive value inoculated with a fungus in containers that are placed in a closed incubation chamber with air flows passed over each container while the chamber is maintained with a predetermined environment of humidity, temperature, carbon dioxide, and oxygen.

The aerial mycelia of the present disclosure are growth products obtained from an inoculated substrate incubated for a period of time (i.e., an incubation time period) in a growth environment, as disclosed herein.

In some embodiments, a method of making an aerial mycelium of the present disclosure comprises placing a growth matrix in contact with a tool. In some embodiments, the tool can have a base having a surface area. In some embodiments, the surface area can be at least about 1 square inch. In some embodiments, the surface area can be at most about 2,000 square feet. In some embodiments, the growth matrix can be placed in contact with the base, e.g., placed on top of or distributed across the base. In some embodiments, the base can be a planar surface. Non-limiting examples of a tool include a tray, a sheet, a table, or a conveyer belt. In some embodiments, the tool can have at least one wall. In some embodiments, the base and the at least one wall can together form a cavity. In some embodiments, the growth matrix can be placed or packed in the tool cavity. In some embodiments, the tool can be an uncovered tool. In some other embodiments, the tool can have a lid, the lid having at least one opening, or the tool can be covered at least in part with a perforated barrier. In some embodiments, the tool may be a perforated base or one with no side walls or a lid, such as a screen or perforated sheet, or a solid sheet (such as a flexible metallic or polymeric sheet) such that a solid substrate may be situated on the base without movement of the substrate material off the side edges or through the base. Non-limiting embodiments of a tool having a lid with an opening are disclosed in US 2015/0033620A1 to Greetham et al., An uncovered tool, or a tool having a lid with an opening or a perforated barrier, and further having growth matrix on or within the tool, can allow for mist to be deposited onto the growth matrix surface, and/or onto any resulting mycelial growth.

“Growth environment” as used herein refers to an environment that supports the growth of mycelia, as would be readily understood by a person of ordinary skill in the art in the mycelial cultivation industry, and which contains a growth atmosphere having a gaseous environment of carbon dioxide (CO₂), oxygen (O₂), and a balance of other atmospheric gases including nitrogen (N₂), and is further characterized as having a relative humidity. In some aspects of the present disclosure, the growth atmosphere can have a CO₂ content of at least about 0.02% (v/v), at least about 5% (v/v), less than about 8% (v/v), less than about 10% (v/v), between about 0.02% and 10%, between about 0.02% and 8%, between about 5% and 10%, or between about 5% and 8%. In some other aspects, the growth atmosphere can have an O₂ content of at least about 12% (v/v), or at least about 14% (v/v), and at most about 21% (v/v). In yet other aspects, the growth atmosphere can have an N₂ content of at most about 79% (v/v). Each foregoing CO₂, O₂, or N₂ content is based on a dry gaseous environment, notwithstanding the growth environment atmosphere relative humidity.

### 1. Temperature and Light

In some embodiments, a method of making an aerial mycelium of the present disclosure comprises incubating the growth matrix in a growth environment, wherein the growth environment has a temperature that supports mycelial growth. In some embodiments, the growth environment has a temperature within a range of about 55° F. to about 100° F., or within a range of about 60° F. to about 95° F. In some more particular embodiments, the growth environment has a temperature within a range of about 80° F. to about 95° F., or within a range of about 85° F. to about 90° F. throughout the incubation time period. In other embodiments, the growth environment has a temperature within a range of about 60° F. to about 75° F., within a range of about 65° F. to about 75° F., or within a range of about 65° F. to about 70° F. In some embodiments, the growth environment temperature can be tuned to improve (e.g., optimize) for the growth of a particular fungal genus, species, or strain.

In some aspects of the present disclosure, the growth environment suitable for the growth of the aerial mycelia of the present disclosure can be a dark environment. “Dark environment” as used herein in connection with a growth environment would be readily understood by a person of ordinary skill in the art in the mycelial cultivation industry and refers to an environment without natural or ambient light, and without growing lights.

Exposing fungi to white light, and especially blue light, has been associated with the induction of fruiting and the enhancement of production efficiency of oyster mushrooms (e.g., see I. Roshita, S. Y. Goh; Effect of exposure to different colors light emitting diode on the yield and physical properties of grey and white oyster mushrooms. AIP Conf. Proc. 9 Nov. 2018; 2030 (1): 020110. https://doi.org/10.1063/1.5066751), the entire contents of which are hereby incorporated by reference in their entirety to the extent not inconsistent with the content of this disclosure. Applicant has discovered that an aerial mycelium for some genera of the present disclosure, such as Ganoderma, absent visible fruiting bodies, can be prepared by the methods of the present disclosure in the presence of white light, which includes blue light. Aerial mycelium prepared in the presence of white light was consistent in yield, thickness, density, morphology and in the absence of visible fruiting bodies when compared to control aerial mycelia produced under the same growth conditions but in a dark environment. Thus, in some embodiments, a growth environment suitable for the growth of the aerial mycelia of the present disclosure is not a dark environment. In some embodiments, the growth environment does not exclude light. In some embodiments, the growth environment can include natural light. In some embodiments, the growth environment can include ambient light. In some embodiments, the growth environment can include a growing light.

### 2. Air Content and Air Flow

As disclosed in US 2015/0033620, environmental conditions for producing a mycological biopolymer include a CO₂ content of about 3% to about 7% (v/v) to prevent full differentiation of the fungus into a mushroom. Accordingly, in some embodiments, the present disclosure provides for methods of producing an aerial mycelium in a growth environment comprising a growth atmosphere, wherein the growth atmosphere can have a CO₂ content within a range of about 0.02% (v/v) to about 10% (v/v), or within a range of about 2% (v/v) to about 4% (v/v). In some embodiments, the growth atmosphere can have a CO₂ content of about 1%, about 2%, about 3%, about 4%, about 5%, about 6%, or about 7% (v/v), or any range therebetween.

Applicant has discovered that an aerial mycelium of the present disclosure can be produced without visible fruiting bodies under conditions wherein mist is introduced into a growth environment having a growth atmosphere containing much lower CO₂ content. For example, Applicant has shown that aerial mycelia obtained from a growth environment of circulating mist and an atmosphere having a mean CO₂ content of about 0.04% (v/v) over the course of the incubation time period or having a mean CO₂ content of about 2% (v/v) over the incubation time period were similar in yield, thickness, density, and morphology to aerial mycelia obtained via growth in an atmosphere having a mean CO₂ content of 5% (v/v) but otherwise identical growth conditions. Thus, the present disclosure advantageously provides for a safer, more efficient, and more cost-effective manufacturing process with reduced environmental impact (e.g., by circumventing the risk of exposure to high CO₂ content growth environments, increasing operator accessibility to growth environments, eliminating the costs associated with CO₂ injection into the growth environment, and reducing off-gassing of CO₂ into the atmosphere). As further disclosed herein, Applicant has shown that aerial mycelia of increased thickness can be obtained via incubation in a growth environment characterized as having a particular misting profile. Prior to this discovery, efforts to obtain thicker aerial mycelia included extending incubation time periods to support continued aerial growth over time. As extended incubation time periods can increase the risk of fruiting body formation, strategies known to attenuate fruiting body formation (e.g., elevated CO₂ content) were simultaneously employed. The present disclosure advantageously provides for methods of making aerial mycelia of increased thickness, absent visible fruiting bodies, by adopting preselected misting profiles, without requiring a high CO₂ content growth environment. The ability to increase aerial mycelial thickness, absent visible fruiting bodies, by tuning mist deposition rate can also advantageously reduce incubation time periods, thereby allowing more efficient production of aerial mycelia and reduced risk of microbial contamination that can occur in high moisture environments.

Thus, the present disclosure provides for a method of growing aerial mycelia in a growth environment comprising a growth atmosphere having markedly reduced CO₂ content compared to the prior state of the art of growing aerial mycelia. Accordingly, in some embodiments, the growth atmosphere CO₂ content can be less than about 3% (v/v). In some embodiments, the growth atmosphere CO₂ content can be no greater than about 2.9% (v/v), no greater than about 2.8% (v/v), no greater than about 2.7% (v/v), no greater than about 2.6% (v/v) or no greater than about 2.5% (v/v). In some further embodiments, the growth atmosphere CO₂ content can be less than 2.5% (v/v). In some embodiments, a growth atmosphere of the present disclosure can have a CO₂ content of at least about 0.02% (v/v). In some embodiments, a growth atmosphere of the present disclosure can have a CO₂ content of at least about 0.03% (v/v). In some further embodiments, the growth atmosphere CO₂ content can approximate ambient atmospheric CO₂ content; for example, the growth atmosphere CO₂ content can be at least about 0.04% (v/v). In some more particular embodiments, the growth atmosphere CO₂ content can be within a range of about 0.02% to about 3% (v/v), about 0.02% to about 2.5% (v/v), about 0.03% to about 3% (v/v), about 0.03% to about 2.5% (v/v), about 0.04% to about 3% (v/v), or about 0.04% to about 2.5% (v/v).

In other embodiments, the growth atmosphere CO₂ content can be within a wider range. Thus, in some embodiments, the growth atmosphere CO₂ content can be within a range of about 0.02% to about 7% (v/v), within a range of about 0.04% to about 7% (v/v), within a range of about 0.1% to about 7% (v/v), within a range of about 0.2% to about 7% (v/v), within a range of about 1% to about 7% (v/v), or within a range of about 2% to about 7% (v/v); or can be within a range of about 0.02% to about 5% (v/v), within a range of about 0.04% to about 5% (v/v), within a range of about 0.1% to about 5% (v/v), within a range of about 0.2% to about 5% (v/v), or within a range of about 1% to about 5% (v/v). In some more particular embodiments, the growth atmosphere CO₂ content can be about 1%, about 2%, about 3%, or any range therebetween. In yet other embodiments, the growth atmosphere CO₂ content can be a mean CO₂ content over the course of the incubation time period. In some embodiments, the growth atmosphere mean CO₂ content can be less than about 3% (v/v), less than 2.5% (v/v), or no greater than about 2% (v/v) over the course of the incubation time period.

It is understood that fungal growth requires respiration, which can increase CO₂ content and decrease oxygen (O₂) content in the growth atmosphere, particularly in an enclosed growth environment such as an incubation chamber or “growth chamber.” In some embodiments, the present disclosure provides for a growth environment having a growth atmosphere that is maintained during the incubation time period by replenishing the growth environment with one or more of the atmospheric gases, such as CO₂, replenishing the growth environment with air having the same composition as the target growth atmosphere composition, venting the growth environment to reduce content of one or more gases, or a combination thereof. In a non-limiting example, if the CO₂ content in a growth chamber is below a target set point, CO₂ gas can be infused into the growth chamber. Conversely, if the CO₂ content exceeds a target set point, then fresh air having the target growth atmosphere composition can be introduced into the growth chamber while venting the chamber to release the existing air having the high CO₂ content. Accordingly, growth chamber atmospheric content can be maintained via CO₂ and fresh air infusion to maintain a target CO₂ set point; as such, O₂ and other atmospheric components are maintained indirectly and fluctuate as a function of fungal respiration. In some other embodiments, the present disclosure provides for a growth environment wherein the growth atmosphere CO₂ and O₂ contents are allowed to modulate with fungal respiration, without adjusting the growth atmosphere to maintain preselected CO₂ or O₂ content. Thus, the growth environment can be a closed system. The present disclosure also provides for a growth environment wherein the growth atmosphere CO₂ and O₂ contents are allowed to modulate with fungal respiration, and further allowing for adjustments to be made to the growth atmosphere under conditions wherein a particular preselected growth atmospheric condition is breached. In a non-limiting example, an aerial mycelium can be grown in a growth atmosphere that allows for natural fungal respiration to occur, with a preselected CO₂ content ranging from about 0.02% to about 7% CO₂ (v/v), wherein the CO₂ content is adjusted (e.g., by injection of CO₂ into the growth atmosphere) if the CO₂ content falls outside the scope of the preselected range.

A growth environment of the present disclosure can be further characterized as having an atmosphere having a pressure as would be readily understood by a person of ordinary skill in the art in the mycelial cultivation industry. In a non-limiting embodiment, a growth atmosphere of the present disclosure can have an atmospheric pressure within a range of about 27 to about 31 inches of mercury (Hg), can have an atmospheric pressure of about 29 to about 31 inches Hg, or can have an atmospheric pressure of about 29.9 inches Hg. In some embodiments, a growth environment of the present disclosure can be characterized as having an ambient atmospheric pressure.

In some aspects of the present disclosure, the growth environment suitable for the growth of the aerial mycelia of the present disclosure is characterized as having an airflow. In some further embodiments, the air composition of the airflow can be substantially the same as the composition of the growth environment atmosphere. In some embodiments, an airflow can be used to direct and/or deposit mist that is present in the growth environment towards or onto a growth matrix. The skilled person can adopt various means of directing the flows of air, including baffles, perforated barriers, airflow boxes and/or other tools that can be suitably positioned in the growth environment or in relation to tools or beds containing growth matrix in order to achieve the desired outcome, including a substantially homogeneous airflow, with respect to direction and/or velocity, across a plurality of growth matrices in the growth environment, and/or a substantially homogeneous introduction and/or deposition of mist in the growth environment.

“Horizontal airflow” as used herein refers to flows of air directed substantially parallel to the surface of a growth matrix and any subsequent extra-particle mycelial growth.

In some other embodiments, preparing an aerial mycelium of the present disclosure can include directing an airflow through the growth environment. In some embodiments, the airflow can be a relatively high airflow environment, wherein the airflow can have a velocity of greater than about 250 linear feet per minute (lfm). In other embodiments, the airflow can be a relatively lower airflow environment, wherein the airflow can have a velocity of less than about 150 lfm, less than about 125 lfm, less than about 100 lfm or less than about 75 lfm. In some more particular embodiments, the growth environment can have an airflow, wherein the airflow velocity is less than about 50 lfm, less than about 40 lfm, less than about 30 lfm or less than about 25 lfm.

In some embodiments, the airflow is a substantially horizontal airflow. In some embodiments, the substantially horizontal air flow can have a velocity of no greater than about 350 lfm, or a velocity no greater than about 300 lfm. In other embodiments, the substantially horizontal airflow can have a velocity of no greater than about 275 lfm, a velocity of no greater than about 175 lfm, a velocity of no greater than about 150 lfm, a velocity of no greater than about 125 lfm, or a velocity of no greater than about 110 lfm. In some further embodiments, the velocity is at least about 5 lfm, at least about 10 lfm, at least about 15 lfm, at least about 20 lfm, at least about 25 lfm, at least about 30 lfm, at least about 35 lfm, at least about 40 lfm, at least about 45 lfm or at least about 50 lfm. In some more particular embodiments, the substantially horizontal airflow has mean velocity of about 5 lfm, about 10 lfm, about 15 lfm, about 20 lfm, about 25 lfm, about 30 lfm, about 35 lfm, about 40 lfm, about 45 lfm, about 50 lfm, about 55 lfm, about 60 lfm, about 65 lfm, about 70 lfm, about 75 lfm, about 80 lfm, about 85 lfm, about 90 lfm, about 95 lfm, about 100 lfm, about 105 lfm, about 110 lfm, about 115 lfm or about 120 lfm. In some more particular embodiments still, the substantially horizontal air flow can have a velocity within a range of about 5 lfm to about 125 lfm, within a range of about 5 lfm to about 100 lfm, within a range of about 5 lfm to about 75 lfm, or within a range of about 5 lfm to about 50 lfm. In yet more particular embodiments, the substantially horizontal air flow can have a velocity within a range of about 5 lfm to about 40 lfm, or within a range of about 5 to about 25 lfm. In other embodiments, the substantially horizontal air flow can have a velocity within a range of about 40 lfm to about 120 lfm. Without being bound to any particular theory, the flows of air can facilitate the distribution of mist throughout the growth environment, can facilitate the distribution of mist onto the growth matrix surface and/or extra-particle mycelial growth, or both. The air flow and misting apparatus can be tuned in concert to achieve the desired mist deposition rate and/or mean mist deposition rate, and to tune the mycelial tissue morphology.

### 3. Mist Deposition

“Mist deposition rate” as used herein refers to the rate at which mist is deposited per discrete instance of mist deposition. Any standalone usage herein of “mist deposition rate,” without the prefix “mean,” refers to the rate at which mist is deposited per discrete instance of mist deposition and is used interchangeably herein with “instantaneous mist deposition rate” or “momentary mist deposition rate.” “Mean mist deposition rate” is not used interchangeably herein with respect to “mist deposition rate” and is as defined elsewhere herein. The mist deposition rate can be based on or determined by measuring the volume of mist deposited on a surface area over a period of time, wherein the period of time is a fraction of the total incubation time period. In a non-limiting example, the mist is deposited on an exposed surface of growth matrix at a mist deposition rate of about 1 μL per square centimeter of growth matrix per hour. In another non-limiting example, the mist is deposited on extra-particle aerial mycelial growth, and the mist deposition rate is about 1 μL per square centimeter of the extra-particle aerial mycelial growth per hour. In some embodiments, the mist deposition rate can be reported as the volume of mist deposited per misting duty cycle. For the purposes of the present disclosure, a mist deposition rate of 1 μL per centimeter squared per hour (1 μL/cm²/hour) is substantially equivalent to a mist deposition rate of 1 milligram per centimeter squared per hour (1 mg/cm²/hour), solute concentration notwithstanding.

“Mean mist deposition rate” as used herein refers to a mist deposition rate averaged over an incubation time period. The mean mist deposition rate can be expressed based on a surface area over which the mist is deposited. In a non-limiting example, the mist is deposited on an exposed surface of growth matrix at a mean mist deposition rate of about a μL per square centimeter of growth matrix per hour. In another non-limiting example, the mist is deposited on an exposed surface of growth matrix containing extra-particle aerial mycelial growth, and the mean mist deposition rate is about 1 μL per square centimeter of the growth matrix containing the extra-particle aerial mycelial growth per hour. For the purposes of the present disclosure, a mean mist deposition rate of 1 μL per centimeter squared per hour (1 μL/cm²/hour) is substantially equivalent to a mean mist deposition rate of 1 milligram per centimeter squared per hour (1 mg/cm²/hour), solute concentration notwithstanding.

In some embodiments, provided is a method of making an aerial mycelium. In some embodiments, the method comprises: providing a growth matrix; and incubating the growth matrix in a growth environment for an incubation time period. In some embodiments, the growth matrix is a fungal-inoculated growth medium. In other embodiments, the growth matrix is a precolonized substrate.

In some embodiments, the aerial growth response can be affected by the presence of mist in the growth environment, and/or by mist deposition in the growth environment, and/or by mist deposition on the growth matrix. Applicant has shown that aerial growth does not occur in the absence of mist in the growth environment and/or in the absence of mist deposition in the growth environment (these conditions give rise to appressed mycelia), and that aerial growth does occur in the presence of mist in the growth environment, and/or in the presence of mist deposition in the growth environment.

In some embodiments, a growth environment can be provided that has an amount of mist present therein. The amount of mist present can be established before or during various actions taken within the growth environment, for example, during incubating a growth matrix. Thus, in some embodiments, a method of making an aerial mycelium of the present disclosure can include exposing a growth matrix to a growth environment that has an amount of mist present therein. In some embodiments, exposing the growth matrix to the growth environment can include introducing mist into the growth environment. In some embodiments, the mist can be introduced into the growth environment resulting in a detectable quantity of deposited mist in the growth environment. In some more particular embodiments, mist can be introduced into the growth environment resulting in a mean mist deposition rate that results in a detectable quantity of deposited mist in the growth environment. For example, mist can be introduced into the growth environment resulting in a mean mist deposition rate that results in a detectable quantity of deposited mist on surfaces of the container or other structure, on the growth matrix, on the extra-particle aerial mycelial growth, and/or on other structures within the growth environment. Methods of detecting deposited mist include visual inspection methods for visibly detectable deposited mist, measuring a quantity of deposited mist based on mass of collected mist or deposited solute, or other reasonable detection methods. A non-limiting example of a method of measuring an amount of deposited mist can be based upon the method of measuring mean mist deposition rate disclosed herein. Thus, in some embodiments, the mist can be introduced into the growth environment resulting in a mean mist deposition rate that does not result in a measurable mass of deposited mist in the growth environment. This can be confirmed after placing one or more open Petri dishes of known surface area in a growth environment during an incubation time period for at least 24 hours and at most about 7 days. Throughout this incubation time period, some amount of mist is present within the growth environment based upon some amount of mist introduction, allowing for the collection of some theoretical amount of deposited mist in the one or more open Petri dishes. The total theoretical mass of collected mist can be determined (to determine the mass of the deposited mist) and divided by the period of time (to determine the mean mist deposition rate based on mass). In embodiments wherein the mist present in the growth environment does not result in measurable mist deposition in the growth environment based on mass, the total amount (i.e., mass) of collected mist is negligible, i.e., not measurable within the tolerance of the balance used to determine the mass, but at some small amount above zero.

In some other embodiments, mist can be introduced into the growth environment resulting in a mean mist deposition rate that does not result in a measurable volume of deposited mist in the growth environment. This can be confirmed after placing one or more open Petri dishes of known surface area in a growth environment during an incubation time period for at least 24 hours and at most about 7 days. Throughout this incubation time period, some amount of mist is present within the growth environment based upon some amount of mist introduction, allowing for the collection of some theoretical volume of deposited mist in the one or more open Petri dishes. The total theoretical volume of collected mist can be determined (to determine the volume of the deposited mist) and divided by the period of time (to determine the mean mist deposition rate based on volume). In embodiments wherein the mist present in the growth environment does not result in measurable mist deposition in the growth environment based on volume, the total amount (i.e., volume) of collected mist is negligible, i.e., not measurable within the tolerance of the volumetric equipment used to determine the volume, but at some small amount above zero.

In yet other embodiments, the mist can be introduced into the growth environment resulting in a mean mist deposition rate that does not result in visible deposited mist in the growth environment. This can be confirmed after placing one or more open Petri dishes in a growth environment during an incubation time period for at least 24 hours and at most about 7 days. Throughout this incubation time period, some amount of mist is present within the growth environment based upon some amount of mist introduction, allowing for the collection of some theoretical amount of deposited mist in the one or more open Petri dishes. During and/or upon completion of the incubation time period, the one or more open Petri dishes can be visually inspected to confirm that no visible amount of mist deposition is present (functionally, when the one or more Petri dishes are dry).

In some embodiments, a growth matrix comprising a colonized substrate may be incubated in a growth environment of the present disclosure. In some embodiments, the colonized substrate may comprise a growth medium pre-colonized with mycelium of a fungus. As used herein, pre-colonization of a growth medium may comprise a discrete incubation phase that spans from inoculation of the growth medium through placement of the colonized growth medium (a growth matrix) in a growth environment.

In some other embodiments, the incubation time period ends no later than when a visible fruiting body forms. In a non-limiting example, the incubation time period can end prior to a karyogamy or meiosis phase of the fungal reproductive cycle. In some other embodiments, the incubation time period ends when a visible fruiting body forms. As disclosed herein, aerial mycelia of the present disclosure can be prepared without the formation of a visible fruiting body, thus, in some embodiments, an incubation time period can end without regard to the formation of a visible fruiting body. Trial incubation runs can be used to inform the period of time in the growth environment during which sufficient extra-particle aerial mycelial growth product occurs (e.g., aerial mycelial growth of a predetermined thickness) without the formation of visible fruiting bodies.

In some embodiments, the method of making an aerial mycelium of the present disclosure can comprise introducing mist into the growth environment throughout the incubation time period.

Beyond the discovery of an aerial growth response to mist, Applicant has also discovered that aerial mycelia of the present disclosure can be prepared by exposing a growth matrix to mist throughout a portion of the incubation time period (e.g., by introducing mist into the growth environment throughout a portion of the incubation time period). For example, introduction of mist to a growing extra-particle aerial mycelium can cause growth of aerial hyphae from the extra-particle aerial mycelium. Applicant has measured vertical expansion kinetics of mycelia over the course of an entire incubation period and has characterized the kinetics as having a primary myceliation phase and a vertical expansion phase. The primary myceliation phase included days 1 to 3 of the incubation time period. Introducing mist throughout a portion of the incubation time period (wherein the portion included the vertical expansion phase), and not introducing mist on days 1 to 3 of the incubation time period was sufficient to produce aerial mycelium having substantially similar characteristics to aerial mycelia obtained by depositing mist throughout the entire incubation period.

Thus, while some embodiments of the present disclosure provide for a method of making an aerial mycelium comprising exposing a growth matrix to a growth environment comprising mist throughout the incubation time period (e.g., by introducing mist into the growth environment throughout the incubation time period, i.e., throughout the entire incubation time period), in other embodiments, the present disclosure provides for a method of making an aerial mycelium comprising exposing a growth matrix to mist throughout a portion of the incubation time period (e.g., by introducing mist into the growth environment throughout a portion of the incubation time period). In some embodiments, a portion of the incubation time period can comprise a vertical expansion phase. In some further embodiments, a portion of the incubation time period can further comprise at least a portion of a primary myceliation phase. In some other embodiments, a portion of the incubation time period can exclude a primary myceliation phase. In yet some other embodiments, a portion of the incubation time period can comprise a vertical expansion phase. Accordingly, in some embodiments, introducing mist into a growth environment throughout a portion of an incubation time period can comprise introducing mist into the growth environment throughout a vertical expansion phase. In some embodiments, introducing mist into the growth environment throughout a portion of the incubation time period can comprise introducing mist into the growth environment throughout a vertical expansion phase and can exclude introducing mist during the primary myceliation phase. In some embodiments, the portion of the incubation time period can terminate at the end of a vertical expansion phase or can terminate at the end of an incubation time period.

In some other embodiments, a portion of an incubation time period can begin during a first day, a second day, a third day or a fourth day of the incubation time period. Accordingly, in some embodiments, introducing mist into a growth environment throughout a portion of an incubation time period can comprise introducing mist into the growth environment during a first, a second, a third or a fourth day of the incubation time period. In some embodiments, the portion of the incubation time period can terminate at the end of a vertical expansion phase or can terminate at the end of an incubation time period.

In some embodiments, the total volume of mist introduced into the growth environment throughout the incubation period, or a portion thereof, is less than about 200 μL/cm², is less than about 100 μL/cm², is less than about 50 L/cm², is less than about 25 L/cm², is less than about 20 L/cm², is less than about 15 μL/cm², or is less than about 10 μL/cm². In some further embodiments, the total volume of mist introduced into the growth environment throughout the incubation period, or a portion thereof, is at least about 5 L/cm².

In some embodiments, the mist can contain one or more dissolved solutes. US 2020/0146224 to Kaplan-Bic et al., the entire contents of which are hereby incorporated by reference in their entirety to the extent not inconsistent with the content of this disclosure, discloses a method of growing a mycelium biopolymer material comprising placing the plurality of containers in a closed incubation chamber and distributing a mist through the incubation chamber for passage over the growth medium in each container, wherein the mist includes moisture and a solute, such as minerals. US 2020/0146224 to Kaplan-Bic et al. further discloses that growing tissue in each container comprises aerial hypha growing up and out of a nutritious space into a non-nutrient environment, and that, in order to control growth in such an environment, the organism employs the use of turgor pressure to regulate the extension of the hyphae at the hyphal tip; thus, regulating the amount, distribution and/or droplet size of available moisture and solutes deposited across the top surface of the growing material can control the osmotic gradient created within the hyphae and subsequently, its growth rate and pattern of colonization. Surprisingly, Applicant has discovered that aerial mycelial growth can be produced by introducing mist into the growth environment, which can result in depositing mist in the growth environment, wherein the mist contains substantially no amounts of dissolved solute onto the growth matrix and/or the extra-particle aerial mycelial growth produced therefrom. In some embodiments, the mist may comprise tap water, distilled water, or water purified by reverse osmosis. The mist may have conductivity. The mist may include one or more solutes which contribute to the conductivity of the mist. In some embodiments, the one or more solutes may include at least one electrolyte.

Thus, in some embodiments, the present disclosure provides for a method of growing an aerial mycelium in a growth environment, the growth environment comprising a growth matrix and mist, wherein the mist can have a conductivity of no greater than about 1,100 μS/cm. In some further embodiments, the mist conductivity can be no greater than about 1000 μS/cm, no greater than about 900 μS/cm, no greater than about 800 μS/cm, no greater than about 700 μS/cm, no greater than about 600 μS/cm, no greater than about 500 μS/cm, no greater than about 400 μS/cm, no greater than about 300 μS/cm, no greater than about 200 μS/cm, or no greater than about 100 μS/cm. In some other embodiments, the mist conductivity can be no greater than about 50 μS/cm, no greater than about 40 μS/cm, no greater than about 30 μS/cm, no greater than about 20 μS/cm, no greater than about 10 μS/cm, or no greater than about 5 μS/cm. In some further embodiments, the method of growing the aerial mycelium comprises introducing mist into a growth environment comprising a growth matrix, wherein the mist can have a conductivity of no greater than about 500 μS/cm. In some further embodiments, the mist conductivity can be no greater than about 400 μS/cm, no greater than about 300 μS/cm, no greater than about 200 μS/cm, or no greater than about 100 μS/cm. In some other embodiments, the mist conductivity can be no greater than about 50 μS/cm, no greater than about 40 μS/cm, no greater than about 30 μS/cm, no greater than about 20 μS/cm, no greater than about 10 μS/cm, or no greater than about 5 μS/cm.

As disclosed herein, in some embodiments, the mist comprises one or more solutes. In some embodiments, the one or more solutes is an additive. Non-limiting examples of additives include mineral nutrients, additives for pH adjustment, additives for electrical conductivity adjustment, soluble sugars, nitrogen sources, or other water-soluble additives. Non-limiting examples of mineral nutrients include calcium chloride, potassium phosphate, magnesium sulfate, ferrous sulfate, manganese sulfate, and copper sulfate. Non-limiting examples of additives for pH adjustment include hydrochloric acid and sodium hydroxide. Non-limiting examples of sugars include glucose, xylose, maltose, maltotriose, and cellobiose. Non-limiting examples of nitrogen sources include peptone, urea, and glutamic acid.

In some further aspects of the disclosure, the mist that is introduced into the growth environment is characterized as having a mist deposition rate and a mean mist deposition rate.

In some embodiments, the mean mist deposition rate is less than or equal to about 10 μL/cm²/hour, is less than or equal to about 5 μL/cm²/hour, is less than or equal to about 4 μL/cm²/hour, is less than or equal to about 3 μL/cm²/hour or is less than or equal to about 2 μL/cm²/hour. In some embodiments, the mean mist deposition rate is less than or equal to about 1 L/cm²/hour, is less than or equal to about 0.95 L/cm²/hour, is less than or equal to about 0.9 μL/cm²/hour, less than or equal to about 0.85 μL/cm²/hour, is less than or equal to about 0.8 μL/cm²/hour, is less than or equal to about 0.75 μL/cm²/hour, is less than or equal to about 0.7 μL/cm²/hour, is less than or equal to about 0.65 μL/cm²/hour, is less than or equal to about 0.6 μL/cm²/hour, is less than or equal to about 0.55 L/cm²/hour, or is less than or equal to about 0.5 μL/cm²/hour. In some further embodiments, the mean mist deposition rate is at least about 0.01 μL/cm²/hour, is at least about 0.02 μL/cm²/hour, is at least about 0.03 μL/cm²/hour, is at least about 0.04 L/cm²/hour or is at least about 0.05 μL/cm²/hour. In yet some further embodiments, the mean mist deposition rate is within a range of: about 0.01 to about 10 L/cm²/hour, about 0.01 to about 5 μL/cm²/hour, about 0.01 to about 4 μL/cm²/hour, about 0.01 to about 3 μL/cm²/hour, about 0.01 to about 2 μL/cm²/hour, about 0.01 to about 1 μL/cm²/hour, about 0.01 to about 0.9 L/cm²/hour, about 0.01 to about 0.8 L/cm²/hour, about 0.01 to about 0.75 μL/cm²/hour, about 0.01 to about 0.7 L/cm²/hour, about 0.02 to about 10 μL/cm²/hour, about 0.02 to about 5 μL/cm²/hour, about 0.02 to about 4 μL/cm²/hour, about 0.02 to about 3 μL/cm²/hour, about 0.02 to about 2 μL/cm²/hour, about 0.02 to about 1 L/cm²/hour, about 0.02 to about 0.9 μL/cm²/hour, about 0.02 to about 0.8 μL/cm²/hour, about 0.02 to about 0.75 μL/cm²/hour, about 0.02 to about 0.7 μL/cm²/hour, about 0.03 to about 10 μL/cm²/hour, about 0.03 to about 5 μL/cm²/hour, about 0.03 to about 4 μL/cm²/hour, about 0.03 to about 3 μL/cm²/hour, about 0.03 to about 2 μL/cm²/hour, about 0.03 to about 1 L/cm²/hour, about 0.03 to about 0.9 μL/cm²/hour, about 0.03 to about 0.8 μL/cm²/hour, about 0.03 to about 0.75 μL/cm²/hour, about 0.03 to about 0.7 μL/cm²/hour, about 0.04 to about 10 μL/cm²/hour, about 0.04 to about 5 L/cm²/hour, about 0.04 to about 4 μL/cm²/hour, about 0.04 to about 3 μL/cm²/hour, about 0.04 to about 2 μL/cm²/hour, about 0.04 to about 1 μL/cm²/hour, about 0.04 to about 0.9 μL/cm²/hour, about 0.04 to about 0.8 μL/cm²/hour, about 0.04 to about 0.75 μL/cm²/hour, about 0.04 to about 0.7 μL/cm²/hour, about 0.05 to about 10 μL/cm²/hour, about 0.05 to about 5 μL/cm²/hour, about 0.05 to about 4 μL/cm²/hour, about 0.05 to about 3 μL/cm²/hour, about 0.05 to about 2 μL/cm²/hour, about 0.05 to about 1 μL/cm²/hour, about 0.05 to about 0.9 μL/cm²/hour, about 0.05 to about 0.8 μL/cm²/hour, about 0.05 to about 0.75 μL/cm²/hour, about 0.05 to about 0.7 L/cm²/hour, about 0.1 to about 10 μL/cm²/hour, about 0.1 to about 5 μL/cm²/hour, about 0.1 to about 4 μL/cm²/hour, about 0.1 to about 3 μL/cm²/hour, about 0.1 to about 2 μL/cm²/hour, about 0.1 to about 1 μL/cm²/hour, about 0.1 to about 0.9 μL/cm²/hour, about 0.1 to about 0.8 μL/cm²/hour, about 0.1 to about 0.75 μL/cm²/hour, about 0.1 to about 0.7 μL/cm²/hour, about 0.2 to about 10 μL/cm²/hour, about 0.2 to about 5 μL/cm²/hour, about 0.2 to about 4 μL/cm²/hour, about 0.2 to about 3 μL/cm²/hour, about 0.2 to about 2 μL/cm²/hour, about 0.2 to about 1 μL/cm²/hour, about 0.2 to about 0.9 μL/cm²/hour, about 0.2 to about 0.8 μL/cm²/hour, about 0.2 to about 0.75 μL/cm²/hour, about 0.2 to about 0.7 μL/cm²/hour, about 0.2 to about 0.6 μL/cm²/hour, about 0.2 to about 0.5 μL/cm²/hour, about 0.2 to about 0.4 μL/cm²/hour, about 0.3 to about 0.5 μL/cm²/hour, about 0.3 to about 0.4 μL/cm²/hour or about 0.30 to about 0.35 μL/cm²/hour. In some more particular embodiments, the mean mist deposition rate is about 0.05 μL/cm²/hour, about 0.10 μL/cm²/hour, about 0.15 μL/cm²/hour, about 0.20 μL/cm²/hour, about 0.25 μL/cm²/hour, about 0.30 μL/cm²/hour, about 0.35 μL/cm²/hour, about 0.40 μL/cm²/hour, about 0.45 μL/cm²/hour, about 0.50 L/cm²/hour, about 0.55 μL/cm²/hour, about 0.60 μL/cm²/hour, about 0.65 μL/cm²/hour, about 0.70 μL/cm²/hour, about 0.75 μL/cm²/hour, about 0.80 μL/cm²/hour, about 0.85 μL/cm²/hour, about 0.90 μL/cm²/hour, about 0.95 μL/cm²/hour, or about 1.0 μL/cm²/hour, or any range therebetween.

In yet some further aspects of the disclosure, the mist that is introduced into the growth environment is characterized as having a mist deposition rate.

In some embodiments, the mist deposition rate is less than about 50 μL/cm²/hour, is less than about 25 μL/cm²/hour, is less than about 15 μL/cm²/hour, is less than about 10 μL/cm²/hour, is less than about 5 μL/cm²/hour, is less than about 4 μL/cm²/hour, is less than about 3 μL/cm²/hour or is less than about 2 μL/cm²/hour. In some more particular embodiments, the mist deposition rate is less than about 1 L/cm²/hour. In some further embodiments, the mist deposition rate is at least about 0.01 μL/cm²/hour, is at least about 0.02 μL/cm²/hour, is at least about 0.03 μL/cm²/hour, is at least about 0.04 L/cm²/hour, or is at least about 0.05 μL/cm²/hour. In yet some further embodiments, the mist deposition rate is within a range of: about 0.05 to about 0.8 μL/cm²/hour, about 0.05 to about 0.75 μL/cm²/hour, about 0.1 to about 0.8 μL/cm²/hour, about 0.1 to about 0.75 μL/cm²/hour, about 0.2 to about 0.8 μL/cm²/hour, about 0.2 to about 0.75 μL/cm²/hour, about 0.2 to about 0.7 μL/cm²/hour, about 0.2 to about 0.6 μL/cm²/hour, about 0.2 to about 0.5 μL/cm²/hour, about 0.2 to about 0.4 μL/cm²/hour, about 0.3 to about 0.5 μL/cm²/hour, about 0.3 to about 0.4 μL/cm²/hour or about 0.30 to about 0.35 μL/cm²/hour. In yet more particular embodiments still, the mist deposition rate is about 0.01 μL/cm²/hour, about 0.02 μL/cm²/hour, about 0.03 μL/cm²/hour, about 0.04 μL/cm²/hour, about 0.05 μL/cm²/hour, about 0.10 μL/cm²/hour, about 0.15 μL/cm²/hour, about 0.20 μL/cm²/hour, about 0.25 μL/cm²/hour, about 0.30 μL/cm²/hour, about 0.35 μL/cm²/hour, about 0.40 μL/cm²/hour, about 0.45 μL/cm²/hour, about 0.50 μL/cm²/hour, about 0.55 μL/cm²/hour, about 0.60 μL/cm²/hour, about 0.65 μL/cm²/hour, about 0.70 μL/cm²/hour, about 0.75 μL/cm²/hour, about 0.80 μL/cm²/hour, about 0.85 μL/cm²/hour, about 0.90 μL/cm²/hour, or about 0.95 μL/cm²/hour, or any range therebetween.

In some embodiments, the mist deposition rate is at most about 20-fold greater than the mean mist deposition rate. In some embodiments, the mist deposition rate is at most about 10-fold greater than the mean mist deposition rate. In some further embodiments, the mist deposition rate is at most about 5-fold greater, is at most 4-fold greater, is at most about 3-fold greater, or is at most about 2-fold greater than the mean mist deposition rate. In some embodiments, the mist deposition rate is substantially the same as the mean mist deposition rate. In some more particular embodiments, the mist deposition rate is less than about 2 μL/cm²/hour and the mean mist deposition rate is less than about 1 μL/cm²/hour. In yet further embodiments, the mist deposition rate and the mean mist deposition rate are each less than about 1 μL/cm²/hour. In yet further embodiments still, the mist deposition rate is less than about 1 μL/cm²/hour, and the mean mist deposition rate is less than about 0.5 μL/cm²/hour.

In other embodiments, the mist deposition rate is at most about 150 μL/cm²/hour, is at most about 100 μL/cm²/hour, is at most about 75 μL/cm²/hour, is at most about 50 μL/cm²/hour, or is at most about 25 L/cm²/hour. In some further embodiments, the mist deposition rate is at least about 10 μL/cm²/hour or is at least about 15 μL/cm²/hour. In some embodiments, the mist deposition rate is at most about 100 μL/cm²/hour, and the mean mist deposition rate is at least about 10 μL/cm²/hour or is at least about 15 μL/cm²/hour.

In some non-limiting embodiments, mist can be introduced into the growth environment via a misting apparatus, which can be incorporated into the growth environment. The apparatus that introduces the mist can be the same or different than an apparatus that controls relative humidity of the growth environment. Non-limiting examples of a misting apparatus suitable for introducing mist into the growth environment include a high pressure misting pump, a nebulizer, an aerosol generator or aerosolizer, a mist generator, an ultrasonic nebulizer, an ultrasonic aerosol generator or aerosolizer, an ultrasonic mist generator, a dry fog humidifier, an ultrasonic humidifier or an atomizer misting system (including but not limited to a “misting puck”), essentially as described in WO 2019/099474A1 to Kaplan-Bie et al., the entire content of which is hereby incorporated by reference in its entirety, or a print head configured to deposit mist, such as a 3D printer, essentially as described in U.S. Patent Publication No. 2020/0157506 to Bayer et al., the entire content of which is hereby incorporated by reference in its entirety. In some other non-limiting embodiments, mist can be introduced into the growth environment via modulation of growth environmental factors such as growth environment atmospheric pressure, temperature and/or relative humidity, or via modulation of the growth atmosphere dew point.

In some embodiments, the mist can be continuously introduced into the growth environment. In some further embodiments, the continuous introduction of mist can be pulse-width modulated (i.e., controlling mist deposition by varying misting apparatus duty cycle and periodicity). In some other embodiments, the continuous introduction of mist deposition can occur at a fixed rate. In yet some other embodiments, the continuous introduction of mist deposition can occur at a variable rate.

In other embodiments, the mist can be intermittently introduced into the growth environment. In some further embodiments, the intermittent introduction of mist can occur at a fixed rate. In other further embodiments, the intermittent introduction of mist can occur at a variable rate. In other further embodiments, the intermittent introduction of mist can occur at regular or irregular periods. In other further embodiments, the intermittent introduction of mist can occur with regular or irregular intervals therebetween without mist introduction.

In some embodiments, a misting apparatus can be operated at a particular duty cycle, a duty cycle defining a percentage of time that the misting apparatus is producing mist. In some embodiments, the misting apparatus is operated at a duty cycle of about 100%. In some embodiments, the misting apparatus is operated at a duty cycle within a range of about 0.1% to about 100%. In some embodiments, the misting apparatus is operated at a duty cycle within a range of about 1% to about 100%, about 5% to about 100%, about 10% to about 100%, about 15% to about 100%, about 20% to about 100% or about 25% to about 100%. In some other embodiments, the misting apparatus is operated at a duty cycle of less than 100%. In some embodiments, the misting apparatus is operated at a duty cycle of no greater than about 75%, no greater than about 50%, no greater than about 40%, no greater than about 30%, no greater than about 25%, no greater than about 20% or no greater than about 15%. In some further embodiments, the misting apparatus is operated at a duty cycle of at least about 1%, at least about 5%, at least about 10%, at least about 15%, at least about 20% or at least about 25%. In some more particular embodiments, the misting apparatus is operated within a range of about 1% to about 15%, about 5% to about 25%, about 25% to about 50%, about 50% to about 75%, or about 75% to about 100%.

In some embodiments, a duty cycle can be further characterized by a cycle period. Non-limiting examples include a duty cycle period of about 3600 second (i.e., about 1 hour), about 1800 seconds (i.e., about 30 minutes), about 360 seconds, (i.e., about 6 minutes), about 180 seconds (i.e., about 3 minutes), about 60 seconds (i.e., about 1 minute), about 45 seconds, about 30 seconds, about 15 seconds, or any value or range therebetween. In some embodiments, the duty cycle period may be between about 15 seconds to about 30 seconds, alternatively between about 30 seconds and about 45 seconds, alternatively between about 45 seconds and about 60 seconds, or alternatively between about 36 seconds and 45 seconds. In some embodiments, a duty cycle period can be at most about 60 minutes, at most about 30 minutes, at most about 15 minutes, or at most about 10 minutes. In some other embodiments, a duty cycle period can be at most about 9 minutes, at most about 8 minutes, at most about 7 minutes or at most about 6 minutes.

As disclosed herein, a method of making an aerial mycelium of the present disclosure can include introducing mist into the growth environment throughout an incubation time period. Introducing mist “throughout the incubation time period” as used herein refers to introducing the mist from the beginning of the incubation time period to the end of the incubation time period. In some embodiments, introducing mist into the growth environment can comprise operating a misting apparatus at a duty cycle of greater than zero from the beginning of the incubation time period to the end of the incubation time period. In a non-limiting example, introducing mist into a growth environment throughout the incubation time period can comprise operating a misting apparatus at a 50% duty cycle from the beginning of the incubation time period to the end of the incubation time period. Further to this non-limiting example, the misting apparatus operating at the 50% duty cycle can have a duty cycle period of at most about 10 minutes. Thus, in this non-limiting example, the misting apparatus can operate (and thus release mist) for 5 minutes out of each 10-minute duty cycle period, and each 10-minute duty cycle period repeats from the beginning of the incubation time period to the end of the incubation time period. Similarly, introducing mist “throughout a portion of the incubation time period” as used herein refers to introducing the mist from the beginning of the portion of the incubation time period to the end of the portion of the incubation time period. In some embodiments, the end of the portion of the incubation time period can be the end of the entire incubation time period. In some embodiments, introducing mist into the growth environment throughout a portion of the incubation time period can comprise operating a misting apparatus at a duty cycle of greater than zero from the beginning of the portion of the incubation time period to the end of the portion of the incubation time period. It will be understood that introducing mist “throughout the incubation time period” and “throughout a portion of the incubation time period” as used herein can include, but do not require, mist introduction at exactly the beginning of, nor exactly the end of the incubation time period or the portion of the incubation time period, for example, in embodiments where the mist is not applied continuously throughout the entirety of the incubation time period or the portion of the incubation time period.

In some embodiments, the present disclosure provides for an mist characterized as having a mean droplet diameter. In some embodiments, the mist has a droplet diameter within a range of about 1 to about 30 microns, within a range of about 1 to about 25 microns, within a range of about 1 to about 20 microns, within a range of about 1 to about 15 microns, within a range of about 1 to about 10 microns, or within a range of about 5 to about 10 microns.

The present disclosure provides for a growth environment atmosphere that is characterized as having a relative humidity sufficient to support mycelial growth. In some embodiments, a growth environment atmosphere of the present disclosure can have a relative humidity of at least about 70%. In some other embodiments, a growth environment atmosphere of the present disclosure can have a relative humidity of at least about 75%, at least about 80%, at least about 85%, or at least about 90%. In yet some other embodiments, a growth environment atmosphere of the present disclosure can have a relative humidity of at least about 95%. In some more particular embodiments, the growth environment atmosphere can have a relative humidity of at least about 96%, or at least about 97%. In some even more particular embodiments, the growth environment atmosphere can have a relative humidity of at least about 98%. In yet more particular embodiments still, the growth environment atmosphere can have a relative humidity of at least about 99% or can have a relative humidity of about 100%. In some embodiments, the growth environment atmosphere can have a relative humidity of 99.0%, 99.1%, 99.2%, 99.3%, 99.4%, 99.5%, 99.6%, 99.7%, 99.8% or 99.9%; or any range therebetween. In some more particular embodiments, the growth environment atmosphere can have a relative humidity of at least 99.9%. In some embodiments, the growth environment atmosphere can have a relative humidity of about 100%. In some further embodiments still, the growth environment atmosphere can have a relative humidity of greater than 100%. In some embodiments, the growth environment atmosphere can be a saturated atmosphere. In some other embodiments, the growth environment atmosphere can be a supersaturated atmosphere. As used herein, a “supersaturated atmosphere” refers to an atmosphere wherein the relative humidity is greater than 100%. Regardless of the relative humidity of the growth environment atmosphere, a growth environment of the present disclosure that is suitable for producing aerial mycelium contains liquid phase water in the form of mist. Thus, even in a growth environment having a saturated or supersaturated atmosphere, methods of growing aerial mycelia of the present disclosure can include introducing mist to the growth environment; accordingly, a growth environment of the present disclosure can include a saturated or supersaturated growth atmosphere plus mist that is introduced from a source other than the water vapor held in the saturated or supersaturated atmosphere. In sum, a growth environment of the present disclosure contains water vapor and droplets of liquid water in the form of mist.

Means of introducing and regulating relative humidity of a growth environment suitable for the growth of mycelia would be readily understood by a person of ordinary skill in the art in the mycelial cultivation industry. In some embodiments, the relative humidity can be controlled independent of misting using conventional heating, ventilation, and air conditioning (HVAC) practices. For example, gaseous moisture can be added to the growth environment by introducing steam into the growth atmosphere via such conventional HVAC practices. In other embodiments, an interplay between the gas phase water vapor and liquid phase mist can be exploited. Accordingly, mist can be introduced into the growth environment at an increased or decreased rate as a means of modifying the growth environment relative humidity.

Applicant has discovered that aerial mycelial growth can be produced from a growth matrix in a growth environment comprising very low levels of mist. Thus, in some aspects of the present disclosure, there is provided a method of making an aerial mycelium, comprising incubating a growth matrix in a growth environment for an incubation time period, wherein the growth matrix comprises a substrate and a fungus; introducing mist into the growth environment throughout the incubation time period, or a portion thereof; and producing extra-particle aerial mycelial growth from the growth matrix; wherein introducing comprises introducing the mist into the growth environment resulting in a mean mist deposition rate of at most about 0.45 μL/cm²/hour. In some embodiments, the introducing comprises introducing the mist into the growth environment resulting in an instantaneous mist deposition rate and the mean mist deposition rate, wherein the ratio of the instantaneous mist deposition rate to the mean mist deposition rate is at most about 20 to about 1. In some embodiments, the ratio of the instantaneous mist deposition rate to the mean mist deposition rate is at most about 10 to about 1. In some embodiments, the ratio of the instantaneous mist deposition rate to the mean mist deposition rate is at most about 5 to about 1. In some embodiments, the introducing further comprises introducing the mist into the growth environment resulting in an instantaneous mist deposition rate of at most about 2 μL/cm²/hour. In some embodiments, the introducing comprises introducing the mist into the growth environment resulting in a mean mist deposition rate of at most about 0.40 μL/cm²/hour, at most about 0.35 μL/cm²/hour, at most about 0.30 μL/cm²/hour, at most about 0.25 L/cm²/hour, at most about 0.20 L/cm²/hour, at most about 0.15 μL/cm²/hour, or at most about 0.10 L/cm²/hour. In some embodiments, the mean mist deposition rate is at least about 0.01 μL/cm²/hour. In some embodiments, the growth matrix comprises the substrate and a fungal inoculum, said fungal inoculum comprising said fungus. In some other embodiments, the growth matrix comprises a colonized substrate, said colonized substrate comprising the substrate, wherein said substrate is pre-colonized with mycelium of said fungus. In some embodiments, the colonized substrate is a fragmented colonized substrate.

In some embodiments, a colonized substrate can be fragmented into smaller portions to provide a fragmented colonized substrate. As used herein, “fragmented colonized substrate” refers to a plurality of discrete particles of colonized substrate. The discrete colonized substrate particles can be characterized as having a particle size. The particle size can have a range, wherein the maximum particle size is less than that of the colonized substrate prior to the fragmentation, and the minimum particle size is substantially the same as the particle size of the substrate prior to the colonization. Methods of fragmenting the colonized substrate can include applying sufficient force to the colonized substrate such that the colonized substrate is fragmented into a plurality of discrete colonized substrate particles. This may simply involve breaking up the colonized substrate into “clumps.” The fragmentation can be performed on a colonized substrate contained in a container. For example, the container can be an aerated bag within which a substrate underwent colonization. Force can be applied to the contained colonized substrate to provide a contained fragmented substrate. Alternatively, the fragmentation can be performed after removal of the colonized substrate from a container. For example, a colonized substrate can reside on an open tray or surface and be physically fragmented, e.g., by hand, machine, or other means of applying force.

In some other aspects of the present disclosure, there is provided a method of making an aerial mycelium, comprising incubating a growth matrix in a growth environment for an incubation time period, wherein the growth matrix comprises a substrate and a fungus; introducing mist into the growth environment throughout the incubation time period, or a portion thereof; and producing extra-particle aerial mycelial growth from the growth matrix; wherein introducing comprises introducing the mist into the growth environment resulting in a mean mist deposition rate of that is below about 0.01 μL/cm²/hour. In some embodiments, the introducing comprises introducing the mist into the growth environment resulting in a mean mist deposition rate that does not result in a detectable quantity of deposited mist in the growth environment. In some embodiments, the introducing comprises introducing the mist into the growth environment resulting in a mean mist deposition rate that does not result in a visible quantity of deposited mist in the growth environment. In some embodiments, the introducing comprises introducing the mist into the growth environment resulting in a mean mist deposition rate that does not result in a measurable quantity of deposited mist in the growth environment. In some embodiments, the introducing comprises introducing the mist into the growth environment resulting in a mean mist deposition rate that does not result in detectable quantities of deposited mist in the growth environment on the growth matrix, the extra-particle aerial mycelial growth, or both. In some embodiments, the growth matrix comprises the substrate and a fungal inoculum, said fungal inoculum comprising said fungus. In some other embodiments, the growth matrix comprises a colonized substrate, said colonized substrate comprising the substrate, wherein said substrate is pre-colonized with mycelium of said fungus. In some embodiments, the colonized substrate is a fragmented colonized substrate.

In some other aspects of the present disclosure, there is provided a method of making an aerial mycelium, comprising incubating a growth matrix in a growth environment for an incubation time period, wherein the growth matrix comprises a substrate and a fungus; introducing mist into the growth environment throughout the incubation time period, or a portion thereof; and producing extra-particle aerial mycelial growth from the growth matrix; wherein the total quantity of mist resulting from the introducing mist that is deposited on the growth matrix, the resulting extra-particle aerial mycelial growth, or both, is negligible. In some embodiments, the growth matrix comprises the substrate and a fungal inoculum, said fungal inoculum comprising said fungus. In some other embodiments, the growth matrix comprises a colonized substrate, said colonized substrate comprising the substrate, wherein said substrate is pre-colonized with mycelium of said fungus. In some embodiments, the colonized substrate is a fragmented colonized substrate.

In some other aspects of the present disclosure, there is provided a method of making an aerial mycelium, comprising providing a growth environment, the growth environment comprising an amount of mist; incubating a growth matrix in the growth environment for an incubation time period, wherein the growth matrix comprises a substrate and a fungus, wherein incubating comprises exposing the growth matrix to the mist during at least a portion of the incubation time period; and producing extra-particle aerial mycelial growth from the growth matrix; wherein a mean mist deposition rate resulting from the amount of mist during the at least a portion of the incubation time period is below about 0.01 μL/cm²/hour. In some embodiments, the mean mist deposition rate is below an amount that results in a detectable quantity of deposited mist in the growth environment. In some embodiments, the method further comprises introducing the mist into the growth environment. In some embodiments, the growth matrix comprises the substrate and a fungal inoculum, said fungal inoculum comprising said fungus. In some other embodiments, the growth matrix comprises a colonized substrate, said colonized substrate comprising the substrate, wherein said substrate is pre-colonized with mycelium of said fungus. In some embodiments, the colonized substrate is a fragmented colonized substrate.

In some other aspects of the present disclosure, there is provided a method of making an aerial mycelium, comprising introducing mist into a growth environment; incubating a growth matrix in a growth environment for an incubation time period, wherein the growth matrix comprises a substrate and a fungus, and wherein incubating comprises exposing the growth matrix to the mist; and producing extra-particle aerial mycelial growth from the growth matrix; wherein introducing comprises introducing the mist into the growth environment resulting in a mean mist deposition rate that is below about 0.01 μL/cm²/hour. In some embodiments, introducing comprises introducing the mist into the growth environment resulting in a mean mist deposition rate that does not result in a detectable quantity of deposited mist in the growth environment. In some embodiments, the growth matrix comprises the substrate and a fungal inoculum, said fungal inoculum comprising said fungus. In some other embodiments, the growth matrix comprises a colonized substrate, said colonized substrate comprising the substrate, wherein said substrate is pre-colonized with mycelium of said fungus. In some embodiments, the colonized substrate is a fragmented colonized substrate.

### DISCUSSION OF THE FIGURES

The following discussion presents detailed descriptions of the several embodiments of apparatus, systems, and methods for growing mycelium. These embodiments are not intended to be limiting, and modifications, variations, combinations, etc., are possible and within the scope of this disclosure.

Mycelium-based textiles, such as mycelium-based leather, are becoming more prevalent as society shifts to more carbon neutral materials. As the demand for mycelium-based textiles increases, supply of aerial mycelium to produce mycelium-based textiles must increase to meet demand. However, aerial mycelium used to create mycelium-based textiles, such as mycelium-based leather, must possess certain material properties. For example, the aerial mycelium must be sufficiently homogeneous to produce a leather that is durable and sufficiently free from undesirable morphologies. Methods of cultivation with improved substrate compositions and environmental conditions are described herein, so as to achieve with improved regularity, homogeneity, total yield, bioefficiency, and morphologies. The methods and substrate compositions provide a scalable method of culturing aerial mycelium that satisfies a desired plurality of material properties, and at scalable yields.

Likewise, mycelium-based protein alternatives are gaining attention as the food industry looks to shift toward more sustainable and carbon-neutral sources of nutrition. As the interest in mycelium-based proteins grows, the production of mycelium for food purposes must increase to meet demand. However, mycelium used for protein alternatives must meet specific quality standards. For instance, the mycelium needs to be uniformly grown to ensure that the resulting food product is not only nutritious but also palatable and free from undesirable textures or tastes. Additionally, cultivation methods that improve substrate compositions and environmental conditions are essential for achieving consistently high-quality, homogeneous mycelium growth. These methods aim to improve aspects such as regularity, homogeneity, yield per cultivation area, bioefficiency, and the overall quality of the mycelium. By developing and applying these advanced cultivation techniques and substrate compositions, a scalable and efficient method of producing mycelium-based protein alternatives can be achieved, meeting the growing demand with products that satisfy rigorous nutritional and sensory quality criteria.

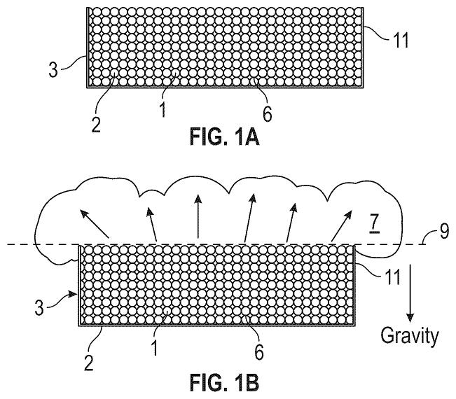

FIG. 1A illustrates an embodiment of a growth matrix 3 suitable to support extra-particle mycelial growth, such as extra-particle aerial mycelial growth. The growth matrix 3 is shown as circles. In some embodiments, the growth matrix 3 can be positioned on (e.g., contained within) a tray (or more generally, any suitable aerial growth tool) 11, such as one with side and bottom walls as shown. The tray as shown does not include a lid (but such can similarly be included if desired). While the tray is shown with the open side facing upward, such configuration is not required, as the tray 11 may just as easily be situated such that the opening be positioned facing sideways or downward (in which configuration the growth matrix 3 would likely be held in place by an unshown textile through which mycelium would grow.

In some embodiments, the tool 11 can have a base having a surface area. In some embodiments, the surface area can be at least about 1 square inch. In some embodiments, the surface area can be at most about 2,000 square feet. In some embodiments, the growth matrix can be placed in contact with the base, e.g., placed on top of or distributed across the base. In some embodiments, the base can be a planar surface. Non-limiting examples of a tool include a tray, a sheet (such as a polymeric web or sheet, or a metallic sheet, which web or sheets may optionally include perforations), a table, a bed, shelf, rack, or a conveyer belt. In some embodiments, the tool can have at least one wall. In some embodiments, the base and the at least one wall can together form a cavity. In some embodiments, the growth matrix can be placed on or packed in a tool having a cavity. In some embodiments, the tool can be an uncovered tool. In some other embodiments, the tool can have a lid, the lid having at least one opening, or the tool can be covered at least in part with a perforated barrier. Non-limiting embodiments of a tool having a lid with an opening are disclosed in US 2015/0033620A1 to Greetham et al.

The growth matrix 3 can include a growth medium and a fungus. For example, the growth matrix 3 can comprise growth medium 2, substrate 1, and colonized (or pre-colonized) substrate 6, to support growth therefrom. The growth matrix 3 can be contained within a growth environment, such as a bioreactor, growth room, or growth chamber. One or more environmental conditions can be controlled within the growth environment, for example, to affect the growth from the growth matrix for desirable results. For example, a processor can be provided to control oxygen (O₂) content, carbon dioxide content (CO₂), temperature, humidity, airflow, misting, and/or other environmental conditions, to, from, and/or within the growth environment, for example, through an interface.

In some embodiments, the growth matrix 3 is implemented without tray 11 (e.g., not shown, but would be positioned on another growth support structure, such as a planar support structure without side walls, such as a mycological growth web).

FIG. 1B illustrates an embodiment of extra-particle aerial mycelial growth 7 from the growth matrix 3 of FIG. 1A. For example, the growth can occur when the growth matrix 3 from FIG. 1A is incubated or otherwise processed within a growth environment under growth conditions suitable for the desired properties of the extra-particle aerial mycelium growth 7 in FIG. 1B. For example, in some embodiments, the environmental condition(s) within the growth environment can be controlled to induce substantially homogeneous extra-particle aerial mycelial growth from the growth matrix.

The extra-particle aerial mycelium growth can extend away from and outward from a surface of the growth matrix to grow an extra-particle aerial mycelium 8, as shown, that will be the basis of separated aerial mycelium. Appropriate growth conditions of the growth matrix 3 in FIG. 1A result in extra-particle aerial mycelium growth initiating across the exposed surface as represented by the directional arrows. Next, extra-particle aerial mycelium growth continues to expand forming a volume of extra-particle mycelial growth 8 as shown in FIG. 1B. The volume of growth can be contiguous. The extra-particle aerial mycelium growth 8 can be grown to various heights. In some embodiments, the growth is about 3-4 inches high above the growth matrix 3. In some embodiments, the growth is considerably higher above the growth matrix, such as at least about 5 inches, or alternatively, at least about 8 inches, or alternatively, at least about 10 inches, or alternatively at least about 12 inches, or alternatively between about 8 and 15 inches, alternatively between about 8 and 12 inches, or alternatively between about 10 and 12 inches, or alternatively, between about 10 and 15 inches above the growth matrix 3. This can be achieved, for example, in up to between about two to three weeks of growth, alternatively in up to about two weeks of growth. It will be understood that although the extra-particle aerial mycelium growth has some amount of irregularity to its upper surface topology as shown (i.e. bumpy or bulbous surface), the drawings are not to scale, and the top surface can be relatively flat. A bumpy or bulbous surface may be one example of heterogeneity of the grown material, whereas a relatively smooth surface may be one example of homogeneity of the grown material).

In some embodiments, the growth can be implemented on a mycological growth web, for example, without the tray 11 shown. The growth web can include the growth matrix and the extra-particle aerial mycelial growth (e.g., without a tray 11). The growth web can include any suitable support structure to support the growth matrix 3 and the extra-particle aerial mycelium growth 8, such as a growing net. The web can be a standard size, such as a 63″W×38′L, 63″W×98′L or any of many other web configurations. Other sizes can be implemented, including lengths up to 90, 100 feet, or more. The growing net can comprise one or more layers of a perforated or nonperforated material, or combinations thereof, such as a plastic, nylon (e.g., nylon weave), or any other flexible, suitable material or multiple layers of material for growing extra-particle aerial mycelium growth 7 from a growth matrix 3. The web can extend in length from right to left in the orientation shown in FIG. 1B.

In some embodiments, the extra-particle aerial mycelium growth 7 and growth matrix 3 can be divided along a separation zone 9 (dot-dashed line) to form a separated aerial mycelium and depleted growth matrix (that is a growth matrix which has supported the growth of at least one flush of aerial mycelium, and which retains some nutritional value). The separation zone 9 (dot-dashed line) can be a zone (e.g., plane) where the extra-particle aerial mycelium growth 8 can be divided and detached from the growth matrix 3. The separation zone 9 need not be linear as shown, although in some embodiments, it can form a plane extending along the dot-dashed lines shown and approximately perpendicular into the view as shown, to form a plane of separation. For embodiments that implement the tray 11, the division of the extra-particle aerial mycelium growth 8 from the growth matrix may result in portions of the extra-particle aerial mycelium growth 8 that extend below the separation zone 9 (such as those shown towards the side edges) to be divided and detached from the separated aerial mycelium.

According to some embodiments, a growth medium, such as the growth medium of the growth matrix 3 may have a nutrition profile. Each component of the growth medium may have a component nutrition profile. For example, the substrate of the growth medium may have a substrate nutrition profile. The nutrition profiles of the growth medium components may be combined into a composite growth medium profile, which defines one or more nutritional values of the growth medium. The nutrition profile of the growth medium may define one or more macronutrients (carbohydrate content, fat content, protein content), one or more micronutrient contents (vitamins or minerals), one or more ingredient amounts, and/or ingredient ratios. Amounts may be defined by weight of the component, a percentage of weight of the substrate made up by the component (w/w), or a percentage of volume made up by the component (v/v). The nutrition profile may be defined based on a dry weight of the growth medium or a hydrated weight of the growth medium. For example, the nutrition profile may define the components of the growth medium before the substrate or growth medium is hydrated. According to at least some embodiments, the nutrition profile may be determined using statistical modeling to identify optimal growth medium constituents and an improved nutrition profile.

The nutrition profile may include values (e.g., target values) for components of the growth medium. These values may be defined as a range of values, a group of values, and/or a particular value. The values may be independently defined or interdependent. In some embodiments, the growth medium nutrition profile may include a non-fiber carbohydrate content (e.g., simple sugar content), a starch content, an ethanol soluble carbohydrate content, a fat content, a protein content, and/or a carbon/nitrogen ratio. In some embodiments, the protein content may include a soluble protein content, an acid detergent insoluble protein content, and/or a neutral detergent insoluble protein content. In some embodiments, the growth medium may include a remainder portion of components that do not contribute to the above value(s) (e.g., target values). In some embodiments, the remainder portion may include a fiber content and/or an ash content. The fiber content may include a lignocellulose content, a cellulose content, and/or a hemicellulose content.

In some embodiments, a prepared growth medium with a nutrition profile may be hydrated such that the growth medium has a moisture content. The moisture content may be at least 60% but less than 70%. In some embodiments, the hydrated growth medium may be sterilized. For example, the hydrated growth medium may be sterilized for 1 hour at 15 psi in a pressure sterilizer.

In one embodiment, the non-fiber carbohydrate content of the nutrition profile may be at least 20% (w/w), at least 25% (w/w), at least 30% (w/w), at least 35% (w/w), at least 40% (w/w), or at least 45% (w/w). In alternative embodiments, the non-fiber carbohydrate content may be greater than 30% (w/w) but less than 35% (w/w). In further alternative embodiments, the non-fiber carbohydrate content may be greater than 30% (w/w) but less than 33% (w/w).

In one embodiment, the starch content may be less than 10% (w/w), alternatively less than 9% (w/w), alternatively less than 8% (w/w), alternatively less than 7% (w/w), alternatively less than 6% (w/w), alternatively less than 5% (w/w), alternatively less than 4% (w/w), alternatively less than 3% (w/w), or alternatively less than 2% (w/w). In a further alternative embodiment, the total starch content may be at least 3% (w/w) but less than 4% (w/w).

In one embodiment, the ethanol soluble carbohydrate content may be at least 1% (w/w), alternatively at least 2% (w/w), alternatively at least 3% (w/w), alternatively at least 4% (w/w), alternatively at least 5% (w/w), alternatively at least 6% (w/w), alternatively at least 7% (w/w), alternatively at least 8% (w/w), alternatively at least 9% (w/w), or alternatively at least 10% (w/w). In a further alternative embodiment, the ethanol soluble carbohydrate content may be at least 2% (w/w) but less than 5% (w/w). In some embodiments, the ethanol soluble carbohydrate content may be determined based on a protein content. For example, the relationship between an ethanol soluble carbohydrate content and a protein content can be used to increase bioefficiency. The ethanol soluble carbohydrate content may be inversely proportional to the protein content. For example, if the protein content is less than 10% (w/w) dry mass, the ethanol soluble carbohydrate content may be less than 5%. If the protein content is greater than 10%, the ethanol soluble carbohydrate content may be less than 3%.

In one embodiment, the fat content may be less than 1% (w/w), alternatively less than 2% (w/w), alternatively less than 3% (w/w), alternatively less than 4% (w/w), alternatively less than 5% (w/w), alternatively less than 6% (w/w), alternatively less than 7% (w/w), alternatively less than 8% (w/w), alternatively less than 9% (w/w), or alternatively less than 10% (w/w). In a further alternative embodiment, the fat content may be at least 1% (w/w) but less than 2% (w/w).

In one embodiment, the protein content may be at least 5% (w/w), alternatively at least 6% (w/w), alternatively at least 7% (w/w), alternatively at least 8% (w/w), alternatively at least 9% (w/w), alternatively at least 10% (w/w), alternatively at least 11% (w/w), alternatively at least 12% (w/w), alternatively at least 13% (w/w), alternatively at least 14% (w/w), or alternatively at least 15% (w/w). In a further alternative embodiment, the protein content may be at least 9% (w/w) but less than 10% (w/w).

In one embodiment, the carbon to nitrogen (carbon/nitrogen) ratio may be at least 20, alternatively at least 25, alternatively at least 30, alternatively at least 35, alternatively at least 40, alternatively at least 45, alternatively at least 50, alternatively at least 55, alternatively at least 60, alternatively at least 65, or alternatively at least 70. In a further alternative embodiment, the carbon/nitrogen ratio may be at least 35 but less than 50. In an alternative embodiment, the carbon/nitrogen ratio may be at least 35 but less than 50. In a further alternative embodiment, the carbon to nitrogen ratio may depend on whether a growth medium is pre-colonized with a fungal inoculum. For example, if the growth medium is pre-colonized with the fungal inoculum, the carbon/nitrogen ratio may be at least 30 but less than 40. If the growth medium is not pre-colonized with the fungal inoculum, the carbon/nitrogen ration may be at least 40 but less than 50.

In some embodiments, a growth cycle may include two or more discrete phases. The two or more discrete phases may include a pre-colonization phase and an aerial growth phase. As discussed above, the pre-colonization phase may span from inoculation of the growth medium to form a growth matrix, incubation of the growth matrix to form a pre-colonized growth matrix, and placement of the pre-colonized growth matrix in a growth environment. The aerial growth phase may span from placement of the pre-colonized growth matrix in a growth environment, growth of an aerial mycelium, and separation of the extra-particle matrix of the aerial mycelium from the growth matrix.

In some embodiments, the growth medium nutrition profile may be different (e.g., modified) between the two or more phases of the growth cycle, relative to each other. The growth medium nutrition profile may be modified by changing the amount and/or types of components, such as by adding substrate or additional amounts or types of nutrients. In some embodiments, the growth medium nutrition profile may be modified by adding at least one nitrogen and/or amino acid sources, such as bone meal, peptone, cercal brans, individual amino acids such as glutamic acid. In some embodiments, the growth medium nutrition profile may be modified by adding at least one mineral, such as calcium, potassium, phosphorus, iron, magnesium, and manganese. In some embodiments, at least one biocontrol treatment may be applied to the growth medium. For example, nematodes may be added to the growth medium for fly control. In another example, a cohabitating bacteria may be added to the growth medium. For example, a pre-colonization growth medium may have a first nutrition profile. Additional substrate, simple sugars and/or nutritional supplements may be added to the pre-colonization growth medium between a pre-colonization phase and an aerial growth phase, modifying the first nutrition profile to generate an aerial growth phase growth medium with a second nutrition profile. In some embodiments, the first nutrition profile may include a first set of values, and the second nutrition profile may include a second set of values.

In some embodiments, the nutrition profile of a pre-colonization phase growth medium may include a protein content, a non-fiber carbohydrate content, a starch content, an ethanol soluble carbohydrate content, a fat content, and/or a carbon/nitrogen ratio. For example, the protein content of the pre-colonization growth medium may be at least 10% but less than 12%. A non-fiber carbohydrate content of the pre-colonization growth medium may be at least 18% but less than 22%. A starch content of the pre-colonization growth medium may be at least 3% but less than 4%. An ethanol soluble carbohydrate content of the pre-colonization growth medium may be at least 1% but less than 5%. A fat content of the pre-colonization growth medium may be at least 1% but less than 5%. A carbon/nitrogen ratio of the pre-colonization growth medium may be at least 25 but less than 35. In some embodiments, the pre-colonization phase growth medium may not include a simple sugar component.

In some embodiments, the pre-colonization growth medium may have a moisture content. The pre-colonization growth medium may be hydrated to achieve the moisture content. For example, the pre-colonization phase growth medium may be hydrated to achieve a moisture content between about 55% and about 75%.

In some embodiments, the moisture content of the pre-colonization phase growth medium may be based on a length of the pre-colonization phase. For example, the pre-colonization phase may be three days or less in length, and the moisture content may be between about 65% and about 75%. The moisture content may be about 70%. In another example, the pre-colonization phase may be four days or more in length, and the moisture content may be between about 55% and about 65%. The moisture content may be about 63%. Advantageously, providing a growth medium with a moisture content based on a length of the pre-colonization phase may increase bioefficiency and ultimate yield of aerial mycelium. Additionally, a longer pre-colonization phase may increase colonization of the pre-colonization phase growth medium by the fungal inoculum, increasing resistance to contamination as theorized below. Further, the moisture content may limit any negative effects caused by variation in the length of the pre-colonization period, e.g. +/−1 day in length.

In some embodiments, the pre-colonization phase growth medium may be different from an aerial growth phase growth medium. The pre-colonization phase growth medium may be modified, e.g., by adding at least one component to the growth medium. The at least one component may modify the nutrition profile of the pre-colonization phase growth medium to reach values of an aerial growth phase growth medium nutrition profile. In some embodiments, the at least one component may be a simple sugar component. The simple sugar component may be one of arabinose, cellobiose, dextrin, dextrose, fructose, fucose, galactose, gentiobiose, glucosamine, glucose, lactose, lactulose, maltodextrin, maltose, maltotriose, mannose, melezitose, melibiose, sucrose, trehalose, xylose, or a combination thereof.

In some embodiments, the nutrition profile of an aerial growth phase growth medium may include a protein content, a non-fiber carbohydrate content, a starch content, an ethanol soluble carbohydrate content, a fat content, and/or a carbon/nitrogen ratio. The protein content of the aerial growth phase growth medium may be at least 9% (w/w). For example, the protein content of the aerial growth phase growth medium may be at least 9% (w/w) but less than 25%. The non-fiber carbohydrate content of the aerial growth phase growth medium may be at least 20% (w/w) but less than 35% (w/w). The starch content of the aerial growth phase growth medium may be at least 3% (w/w) but less than 4% (w/w). The ethanol soluble carbohydrate content of the aerial growth phase growth medium may be at least 2% (w/w) but less than 5% (w/w). The fat content of the aerial growth phase growth medium may be less than 2% (w/w). The carbon/nitrogen ratio of the aerial growth phase growth medium may be at least 30 but less than 40. In some embodiments, the nutrition profile may include a remainder portion in an amount that is relative to the values. The remainder portion may be determined as the remaining percentage of the growth medium not defined by the value(s).

Advantageously, the pre-colonization phase may reduce the amount of spawn needed to grow an aerial mycelium. The pre-colonization phase, a pre-colonization phase growth medium nutritional composition, the modification of the pre-colonization nutrition profile prior to an aerial growth phase, or a combination thereof may increase the bioefficiency and ultimate mass yield of the aerial mycelium. In some embodiments, a simple sugar component may be added to a pre-colonization growth matrix after the pre-colonization phase. The absence of the simple sugar component during pre-colonization and/or subsequent modification of the pre-colonization growth matrix nutrition profile to include the simple sugar component can increase the bioefficiency and mass yield of the aerial mycelium. Further, the pre-colonization phase, a pre-colonization phase growth medium nutritional composition, the modification of the pre-colonization nutrition profile prior to an aerial growth phase, or a combination thereof may increase the accessibility and digestibility of nutritional compounds. For example, the pre-colonization phase may increase the availability of acid detergent insoluble protein content, which may not be digested or consumed by a mycelium without the pre-colonization phase. In turn, a broader scope of components can be used to create a growth medium, and other component may be removed.

The pre-colonization phase may also increase resistance to contamination during a growth cycle. During the pre-colonization phase, the fungal inoculum may colonize the pre-colonization growth medium. It is theorized that the colonization of the pre-colonization growth medium by the fungal inoculum during the pre-colonization period allows the fungal inoculum to achieve a priority effect that allows the fungal inoculum to outcompete and eliminate would-be contaminants during the aerial growth phase, such as bacteria. It is also theorized that the priority effect reduces the need of asepsis and maintaining a sterile environment during handling and loading the pre-colonization phase growth matrix into the tool and/or the growth environment. Further, the modification of the pre-colonization phase growth medium with a simple sugar component after the pre-colonization period may increase the bioefficiency of the aerial mycelium.

In some embodiments, growth medium performance may also be analyzed using one or more growth medium metrics. The growth medium metrics may include a bioefficiency and a yield by area metric. Bioefficiency may be defined by ratio of a dry mass (DM) of the aerial mycelium to the dry mass of the growth medium. The yield by area metric may be defined by a ratio of the dry mass of the aerial mycelium to the area of the tool, such as the tool 11.

According to some embodiments, the growth medium with a particular nutrition profile may be used to culture an aerial mycelium with desirable, pre-determined material properties. In some embodiments, a pre-colonization phase growth medium with a first nutrition profile and an aerial growth phase growth medium with a second nutrition profile may be used to culture an aerial mycelium with desirable, pre-determined material properties. The pre-colonization phase growth medium and the aerial growth phase growth medium may be prepared to have the particular nutrition profile, such as the pre-colonization phase growth medium nutrition profile described above, and the aerial growth phase growth medium nutrition profile described above.

In some embodiments, the pre-colonization phase growth medium may be inoculated with a fungal inoculum. The pre-colonization phase growth medium may be inoculated at an inoculation rate. The inoculation rate can be expressed as a percentage of the volume of the substrate or growth medium, such as (wet mass spawn/dry mass growth medium). In some embodiments, the inoculation rate may be determined based on the nutrition profile of the dry mass of the aerial growth phase growth medium. For example, fungal inoculum may be a Ganoderma sessile inoculum. The pre-colonization phase growth medium may be inoculated with the Ganoderma sessile inoculum at an inoculation rate of about 15% (wet mass spawn/dry mass aerial growth phase growth medium).

The inoculated pre-colonization phase growth medium may be referred to as a pre-colonization growth matrix. The pre-colonization growth matrix may be placed in a pre-colonization receptacle. The pre-colonization receptacle may receive the pre-colonization growth matrix and support the pre-colonization growth matrix during the pre-colonization phase. The pre-colonization receptacle may be one of a bag, a bed, a tray, a table, a growth web, a conveyor belt, etc. For example, the pre-colonization receptacle may be a filter-patch bag, a plastic bag, or other receptacle. The pre-colonization growth matrix may be incubated in the pre-colonization receptacle for a pre-colonization period. In some embodiments, the pre-colonization period may be at least 24 hours but less than 168 hours. For example, the pre-colonization period may be at least 72 hours but less than 168 hours. The pre-colonization period may be about 72 hours.

The pre-colonization receptacle (e.g., a filter patch bag) containing the pre-colonization growth matrix may be kept within a pre-colonization growth environment. The pre-colonization growth environment may support the colonization of the pre-colonization growth medium by the fungal inoculum during the pre-colonization phase. The pre-colonization growth environment may include a target temperature, a target light exposure, and/or other environmental conditions described herein. For example, in some embodiments, the pre-colonization growth matrix may be kept at room temperature and exposed to ambient light during the pre-colonization period. In another example, the pre-colonization growth matrix may be kept at room temperature and be kept in the dark during the pre-colonization period, i.e. may not be exposed to light.

After the pre-colonization period has elapsed, the pre-colonization growth matrix may be removed from the pre-colonization receptacle. In some embodiments, the pre-colonization growth matrix may be processed after being removed from the pre-colonization receptacle. Processing the pre-colonization growth matrix may include chemical modifications, such as modifying a nutrition profile of the pre-colonization phase growth medium and/or mechanical modifications, such as grinding, mixing, compressing (e.g., by rolling), wetting and/or other modifications. For example, the pre-colonization growth matrix may be compressed to achieve a packing density. In another example, the pre-colonization growth matrix may be wetted to achieve a moisture content. In some embodiments, a simple sugar component may be added to the pre-colonization growth matrix after the pre-colonization period has elapsed. In some embodiments, the pre-colonization growth matrix may be ground, i.e. reduced to discrete particles, after being removed from the pre-colonization receptacle but before being placed into the tool 11. For example, the pre-colonization growth matrix may be removed from the pre-colonization receptacle, ground, and a simple sugar component may be added, modifying the pre-colonization phase growth medium nutrition profile to meet the values of the nutrition profile of an aerial growth phase growth medium.

The pre-colonization growth matrix may be loaded into a tool, such as the tool 11. In some embodiments, the pre-colonization growth matrix may be loaded into the tool, and the second un-inoculated portion of the growth medium may be layered on top of the first portion to create a “casing layer” as used herein. A casing layer may comprise a layer of organic or inorganic material. A casing layer may be placed on top of and/or below an inoculated growth medium. In some embodiments, an inoculated growth medium can comprise a patterning of spawn. A casing layer can be made of, for example, vermiculite, peat moss, coconut coir, any material that can be used as an uninoculated substrate, an uninoculated growth medium, or a combination of such materials. For example, the casing layer may include a pre-colonization phase growth medium or an aerial growth phase growth medium.

In some embodiments, the casing layer may comprise a growth medium with a third nutrition profile that may be differentiated from the pre-colonization phase growth medium and/or the aerial growth phase growth medium. In some embodiments, the casing layer may include the nutrition profile of the pre-colonization phase growth medium or the aerial growth phase growth medium, but the casing layer may be comprised of one or more components, where at least one component is differentiated from the pre-colonization phase growth medium or the aerial growth phase growth medium. In some embodiments, the casing layer may comprise one or more properties, and at least one of the properties may be differentiated from the properties of the pre-colonization phase growth medium or the aerial growth phase growth medium. For example, the casing layer may have a different particle size, packing density, and/or moisture content. A casing layer can be included above or below an inoculated growth medium or both above and below an inoculated growth medium. In some embodiments a casing layer can serve as a means for controlling the topology of an aerial mycelium.

In some embodiments, the casing layer may comprise an aerial growth phase growth medium. The aerial growth phase growth medium may include an aerial growth phase nutrition profile, such as the aerial growth phase growth medium nutrition profile described above. In some embodiments, the aerial growth phase growth medium may be positioned to form a casing layer on a pre-colonization growth matrix. The pre-colonization growth matrix and casing layer may be referred to as an aerial growth phase growth matrix. In some embodiments, the casing layer size may be described as a weight percent of the aerial growth phase growth matrix. In some embodiments, the casing layer may weigh from about 5% (w/w) to about 25% (w/w) of the aerial growth phase growth matrix. In one example, the casing layer may be 10% (w/w) of the aerial growth phase growth matrix. In another example, the casing layer may be 25% (w/w) of the aerial growth phase growth matrix. In some embodiments, the casing layer size may include a thickness. The thickness of the casing layer may be between about 0.1 cm to about 3 cm. For example, the casing layer may be 0.5 cm thick according to some embodiments. The 0.5 cm thick casing layer may be 10% (w/w) of the aerial growth phase growth matrix. In another example, the casing layer may be 1 cm thick. The 1 cm thick casing layer may be from about 20%-to about 25% (w/w) of the aerial growth phase growth matrix.

Advantageously, the casing layer may make the fungal inoculum and the aerial mycelium more durable and resistant changes within a growth environment. For example, as demonstrated by the results from Example 1, the casing layer made the fungal inoculum and aerial mycelium more resilient to higher CO₂ variability during an aerial growth phase. In turn, the aerial mycelium can be grown more efficiently and consistently in larger growth environments, which may have higher internal variability than small scale environments.

The pre-colonization growth matrix may be loaded into a growth environment for culturing. In some embodiments, loading the pre-colonization growth matrix into the growth environment may end a pre-colonization phase and begin an aerial growth phase. The growth environment supports the growth of aerial mycelium as described above. In some embodiments, the environmental conditions within the growth environment may be controlled by one or more processors. The one or more processors may maintain target environmental conditions. The target environmental conditions may be ranges, series of values, or specific values. According to at least some embodiments, the one or more processors may be placed in electronic communication with one or more sensors disposed within the growth environment. Based on inputs from the one or more sensors, the one or more processors may adjust the environmental conditions to comply with the target values. The environmental conditions may include a temperature of the growth environment, a CO₂ content of the growth atmosphere of the growth environment (or other gases), an airflow velocity, a mist deposition rate, and a mist composition. The growth matrix may be loaded into a tool, such as the tool 11, at a packing density. In some embodiments, the packing density may be defined by the dry mass of the growth matrix per volume. The packing density may be greater than 1 g/in³ but less than 6 g/in³. For example, the packing density may be between 4 g/in³ and 6 g/in³. In some embodiments, the packing density may be determined based on the components of the growth medium. The tool 11 may then be placed within a growth environment to be cultured for an incubation period. The incubation period may be at least 7 days.

During the incubation period, the growth environment may control one or more environmental conditions such as a temperature of the growth environment, a CO₂ content of the growth atmosphere of the growth environment, an airflow velocity, a mist deposition rate, a mist composition, and a mist duty cycle. In some embodiments, the one or more environmental conditions may conform to static values and/or ranges. In some embodiments, the one or more environmental conditions may vary during an incubation period. The one or more environmental conditions may be set to an initial value which increases or decreases passively during an incubation period. In some embodiments, the one or more environmental conditions may be periodically or continuously modified. The modification of the one or more environmental conditions may be in response to the growth of the aerial mycelium, one or more properties of the aerial mycelium, and/or one or more properties of the growth matrix (e.g., a pH of the growth matrix). For example, at least one environmental condition may be modified after a primary myceliation phase during an aerial growth phase. In some embodiments, the modification of the one or more environmental conditions may occur after a set time period has elapsed. In some embodiments, the modification of the one or more environmental conditions may be automatic.

During the incubation period, the temperature of the growth environment may be maintained at 30° C. with a standard deviation of about +/−0.4° C. The CO₂ level may be maintained such that the mean CO₂ level throughout the incubation period is about 2.34% with a maximum standard deviation over the incubation period of less than 1.5%. In some embodiments, the CO₂ level may be increased during a first portion of the incubation period until the CO₂ level reaches a target CO₂ level. For the second, subsequent portion of the incubation period the target CO₂ level may be maintained. The increase in CO₂ levels over time may be referred to as a “ramp rate,” which is defined as a percentage increase in CO₂ over a period of time. For example, during the first portion of the incubation period, the CO₂ ramp rate may be at least 0.4% per hour. In some embodiments, the CO₂ may reach 50% of the target CO₂ level before a target time within the first portion of the incubation period. For example, the target time for the growth environment to reach 50% CO₂ target level before at least 20 hours.

In some embodiments, the airflow velocity may be maintained around the tool 11 such that the mean airflow velocity throughout the incubation period is greater than 40 ft/min but less than 105 ft/min. In some embodiments, the airflow velocity across the aerial mycelium may increase during the aerial growth phase. This increase may be caused by the displacement of air in the growth environment by the extra-particle aerial mycelial growth, leading to an increase in airflow through a smaller volume. In some embodiments, the airflow velocity may change during two or more portions of the aerial growth phase. For example, to account for the displacement of air within the growth chamber by the aerial mycelium, a first airflow velocity may be maintained for a first portion of the aerial growth phase, and a second airflow velocity may be maintained for a second portion of the aerial growth phase. The first airflow velocity may be greater than 40 ft/min but less than 60 ft/min. The second airflow velocity may be between 60 ft/min and 110 ft/min.

In some embodiment the mist deposition rate may be maintained throughout the incubation period such that the mean mist deposition rate is at least 0.1 mg/cm²/hour but less than 0.5 mg/cm²/hour. For example, the mean mist deposition rate may be about 0.25 mg/cm²/hour. In some embodiments, mist may be introduced into the growth environment with an ultrasonic misting puck. The ultrasonic misting puck may operate on at least a 90% duty cycle over a 45 second operation period. In some embodiments, the mist deposition rate may change during two or more portions of the aerial growth phase. The mist deposition rate may be a first value for a first portion of the aerial growth phase and may be a second value during a second portion of the aerial growth phase. For example, the mist deposition rate may be 0.4 mg/cm²/hour for a first portion of the aerial growth phase, and after the first portion of the aerial growth phase has elapsed, the mist deposition rate may be increased to 0.5 mg/cm²/hour.

In some embodiments, the composition of the mist and/or the properties of the mist may be controlled. For example, the mist may be comprised of tap water with a minimum conductivity of at least 250 μS/cm. In some embodiments, the conductivity of the mist may change during two or more portions of the aerial growth phase. The mist conductivity may be a first value during a first portion of the aerial growth phase. and may be a second value during a second portion of the aerial growth phase. In some embodiments, the mist conductivity may be modified by altering one or more mist solutes. The one or more mist solutes may include at least one electrolyte. For example, the mist conductivity may be a first value during a first portion of the aerial growth phase, and at least one electrolyte may be added or removed from the mist. The addition or removal of the at least one electrolyte may alter the mist conductivity to a second value, and the mist conductivity may be maintained at the second value during a second portion of the aerial growth phase.

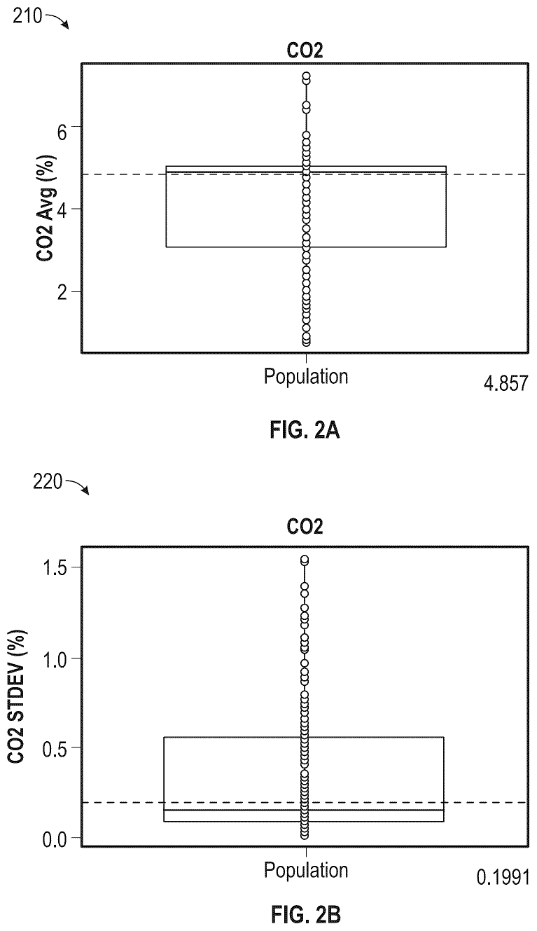

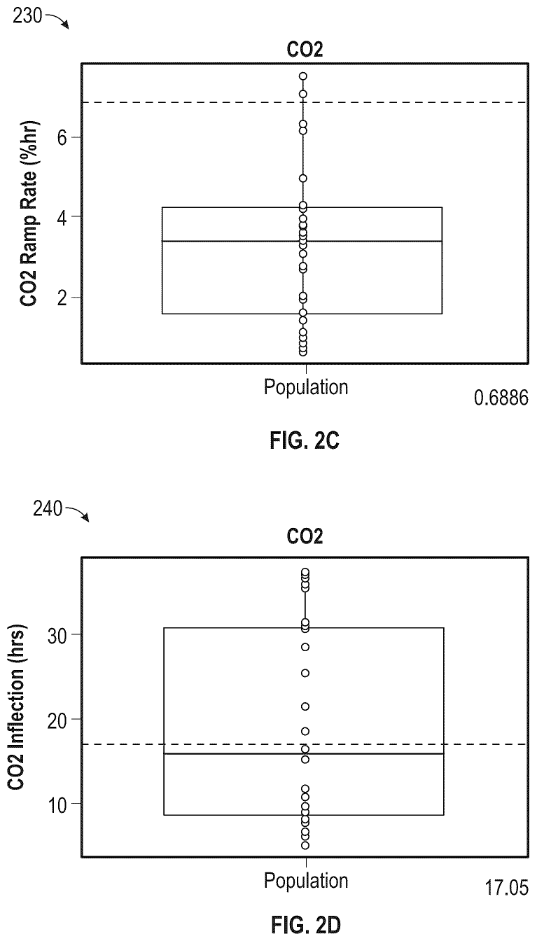

FIGS. 2A-D are illustrative plots showing the optimum CO₂ parametrization against the full experimental distribution evaluated. FIG. 2A-B show horizontal bars to illustrate the optimum and threshold setpoints for mean environmental CO₂ 210 as well as the variance (standard deviation) 220 over the whole incubation period. Additionally, FIGS. 2C-D show CO₂ ramp up parameters as specified, including the ramp rate (i.e., the slope of CO₂ increase over time) 230 and the inflection point (the time at which 50% of the mean CO₂ level is achieved) 240. In some embodiments, deviation from the specified upper or lower thresholds may result in significant effects on growth quality.

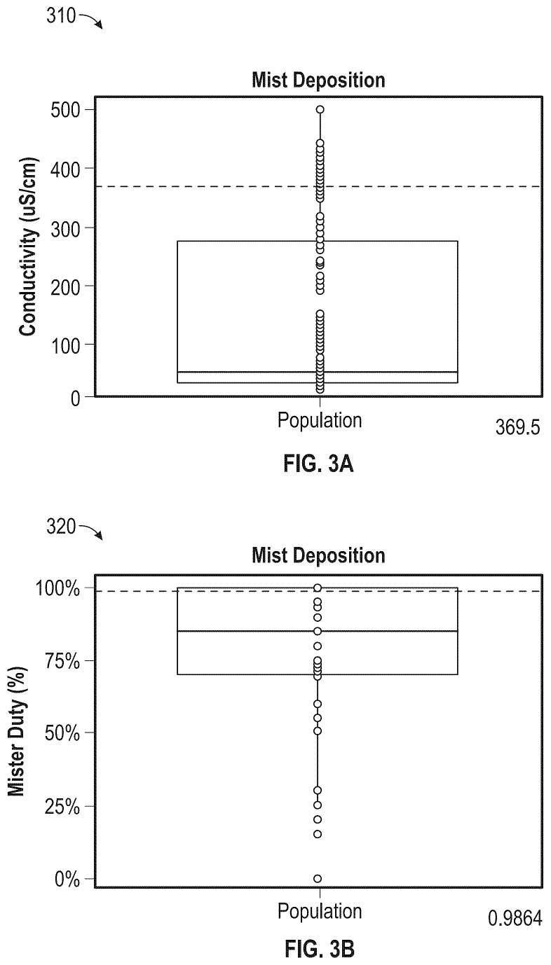

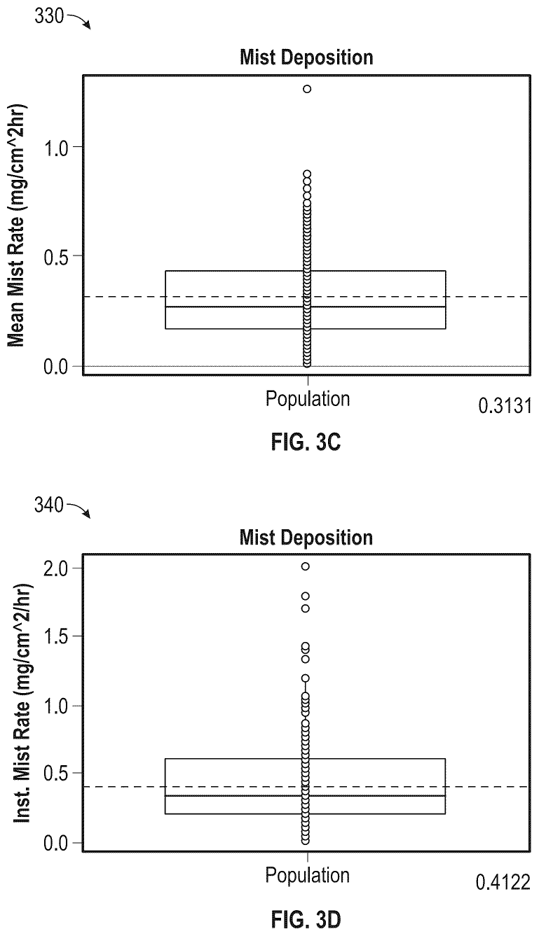

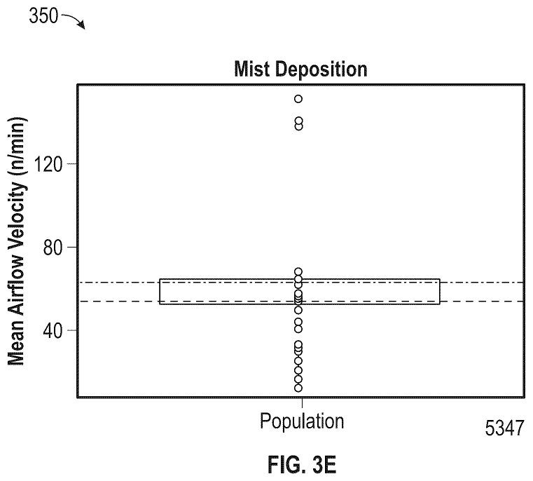

FIGS. 3A-E are illustrative plots showing the optimum mist deposition parametrization against the full experimental distribution evaluated. FIGS. 3A-D show horizontal bars at the optimum and threshold setpoints for mist water conductivity 310, misting apparatus periodicity/duty (where duty fraction over a given duty period defines the periodicity of mist application to the mycelium) 320, mean mist deposition rate 330, instantaneous mist rate 340, and mean airflow velocity over the whole incubation period 350. FIG. 3E shows data on mean airflow velocity. In some embodiment, deviation from the specified upper or lower thresholds may result in significant effects on growth quality.

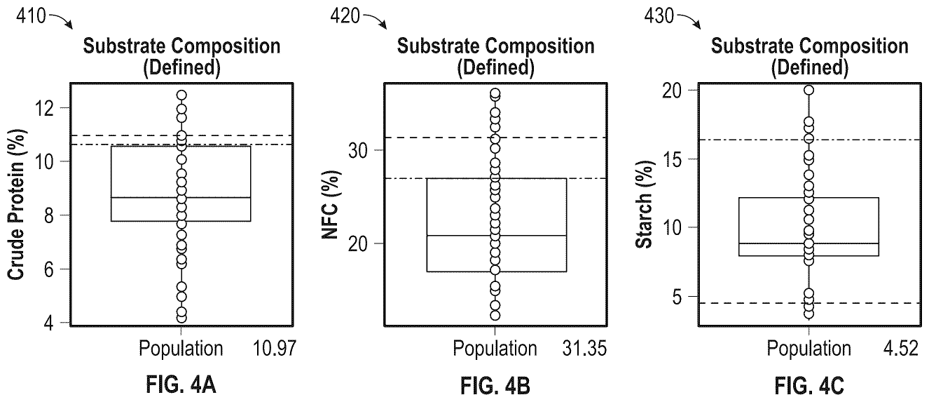

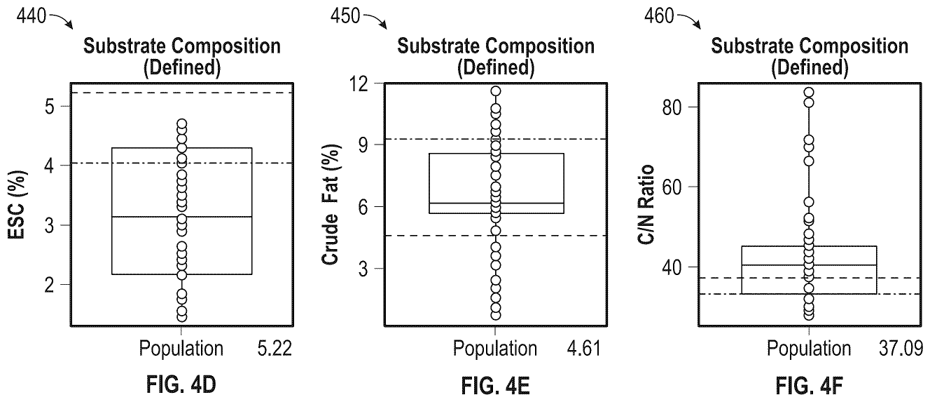

FIGS. 4A-F are illustrative plots showing the optimum substrate composition with respect to individual components thereof. Horizontal bars describe the optimum and threshold setpoints for crude protein 410, non-fiber carbohydrates 420 (NFC), starch 430, ethanol soluble carbohydrates 440, crude fat 450 and carbon to nitrogen ratio 460.

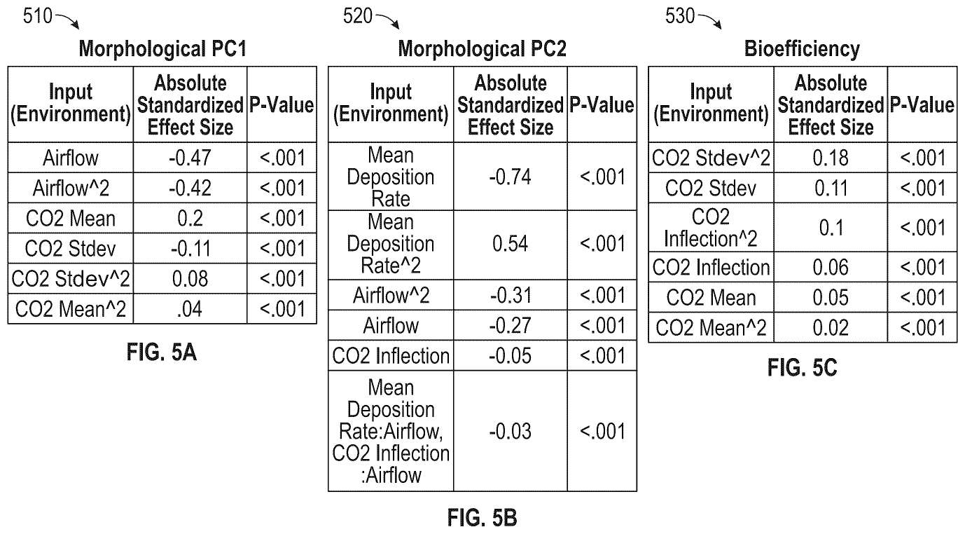

FIGS. 5A-C and 6A-C show the ranked effects between the environmental and substrate nutrition parameters, where morphological principle component 1 (PC1) and morphological principle component 2 (PC2) describe morphological growth quality, and bioefficiency describes the dry mass conversion efficiency of substrate to mycelium. The higher the absolute effect size the more sensitive the given growth quality is to the given environmental or nutritional parameter. Sensitivity of parameters is ranked by the absolute standardized effect size a function of variance around the optimum (i.e., the higher the absolute effect size the more sensitive an output is to the given input). Second-order relationships (i.e. indicating curvature in the response) are denoted with superscript two (‘2’) and interactivities between multiple inputs are denoted with a colon (‘:’). Inputs not shown have effect sizes approaching 0. The environmental parameters 520-530 and the nutritional profile parameters 610-630 may be configured to support the growth of an aerial mycelium with one or more material properties. The one or more material properties may include morphological segmentation, a PC1 value, a PC2 value, and a density value. The morphological segmentation value may be determined using image analysis methods. The image analysis may comprise capturing a 3D topological image of a region of the aerial mycelium, segmenting the image into discrete regions, and determining a morphological segmentation value based on a number of segments. Image analysis may be used to analyze morphological properties of the aerial mycelium, such as directional bias of growth.

The PC1 and PC2 values may be derived from separate principal component analysis of a plurality of properties of the aerial mycelium such as volume, height, density, morphological segmentation, and topological variance. PC1 may be determined based on a principal component analysis of the height, volume, and degree of morphological segmentation of the aerial mycelium. PC2 may be determined based on a principal component analysis of topological variance and directional bias of growth. Representative variables may be identified via the squared cosign values within the principal component analysis, as would be understood by the skilled artisan in the mycelium cultivation industry. In some embodiments, increasing value in both PC1 and PC2 may be associated with (1) increasing volume, (2) reducing topological variance, and (3) fewer discrete morphological segments, which represent a more homogeneous growth quality that is desirable for producing a mycelium-based textile. As shown in FIG. 5A-B, PC1 represents approximately 40% of total morphological variance and describes the responsiveness of mycelium growth height, the absolute variance of growth height (i.e., the standard deviation and the residual standard error of the vertical dimension), total volume, and the number and size of discrete morphological features (i.e., ‘bulbs’). Increasing values of PC1 correspond with increasing height and volume with increasing absolute variance and fewer larger discrete morphological features. PC2 represents approximately 12% of total morphological variance and describes the coefficient of variation of the vertical dimensions (i.e., the surface roughness normalized to growth height), where decreasing values of PC2 are associated with increasing surface roughness normalized by height. The remainder of morphological variance (approximately 48%) is mostly captured in morphological information that is not relevant to mycelial growth and uniformity.

The aerial mycelium grown with these example pre-colonization phase and aerial growth phase growth medium compositions and with these environmental conditions may comply with one or more target material properties. For example, the aerial mycelium may satisfy a target density value, a target PC1 value, and/or a target PC2 value. Moreover, aerial mycelium that complies with the one or more target material properties may be reliably produced using the same growth medium composition and environmental conditions. In turn, production of aerial mycelium can be scaled to satisfy an increasing demand while assuring a quality end product, such as a mycelium-based leather. In some embodiments, modeling techniques may be implemented to improve the growth medium composition and/or environmental conditions of the growth environment to produce an aerial mycelium with the one or more material properties.

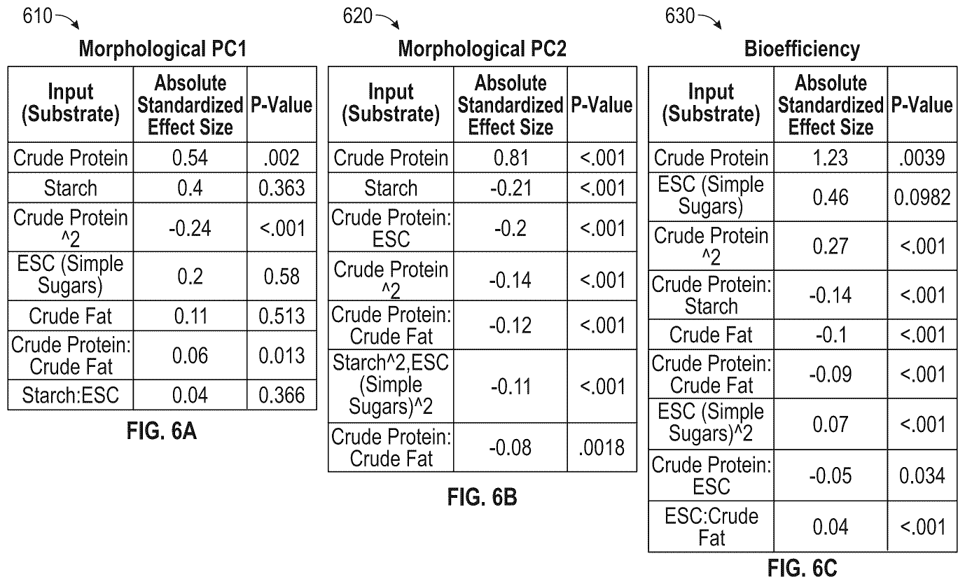

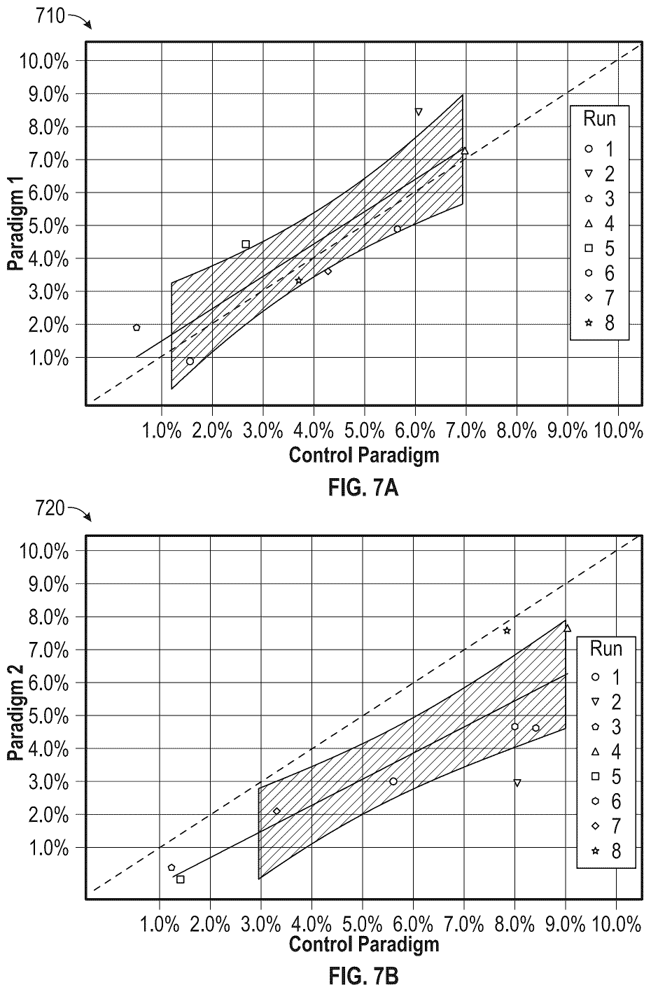

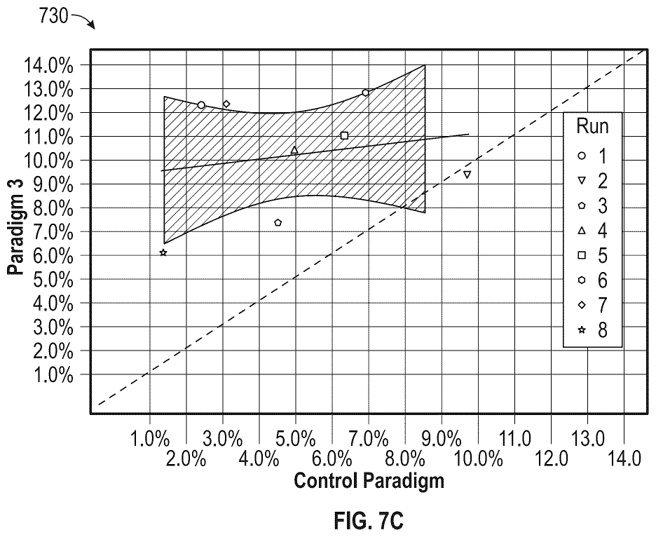

FIGS. 7A-C depict cultivation paradigm model improvements for three experiments. Model improvement experiments 710, 720 and 730 considered three variations of a cultivation paradigm against the control paradigm. The plots illustrate the linear fit between pairs of substrate treatments for a given paradigm as compared to a control paradigm. FIG. 7A illustrates results for Paradigm 1 (1-day pre-colonization) 710, where data were not meaningfully different from the control paradigm. FIG. 7B illustrates results for Paradigm 2 (3-day pre-colonization) 720, where data demonstrated a significant negative effect on bioefficiency independent of substrate composition. FIG. 7C illustrates results for Paradigm 3 (3-day precolonization and backloading simple sugars) 730, where data demonstrated a significant positive effect on bioefficiency over the control with potential interactivities with substrate composition.

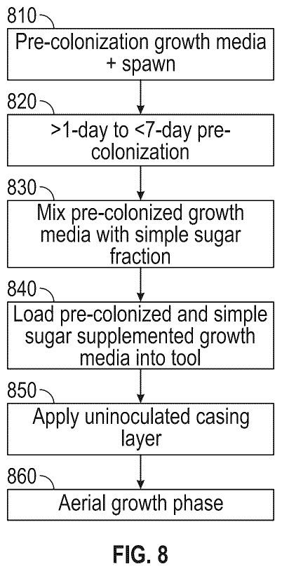

FIG. 8 shows a flow diagram describing the process of cultivating aerial mycelium where the pre-colonization growth medium is prepared and inoculated with spawn 810, is incubated (pre-colonization phase) 820, is prepared with supplemental nutrition 830, is loaded into the tool 840, an uninoculated casing layer fraction is applied to the pre-colonized substrate 850, and is placed in an aerial growth phase 860.

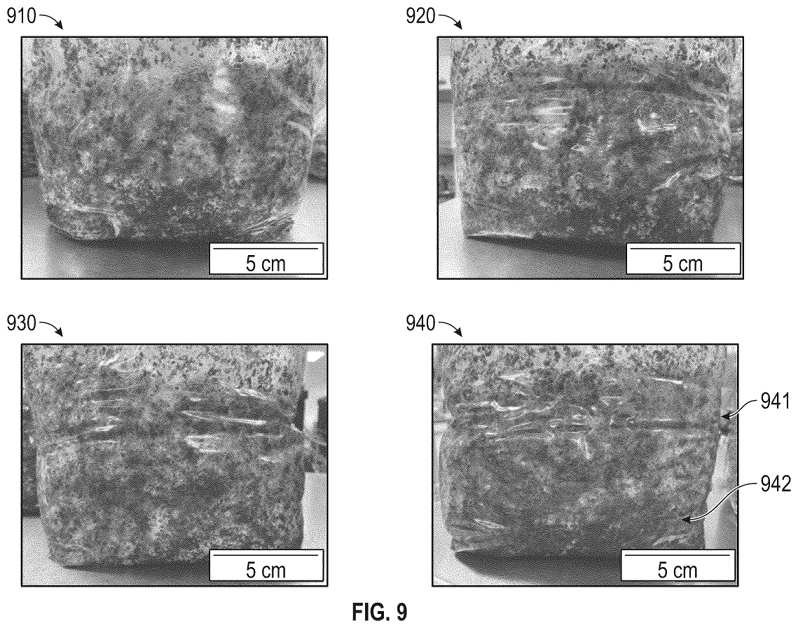

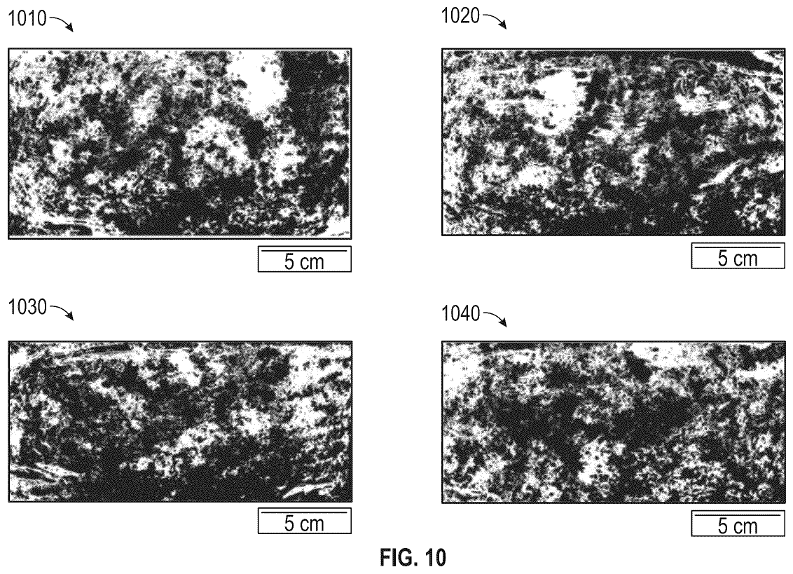

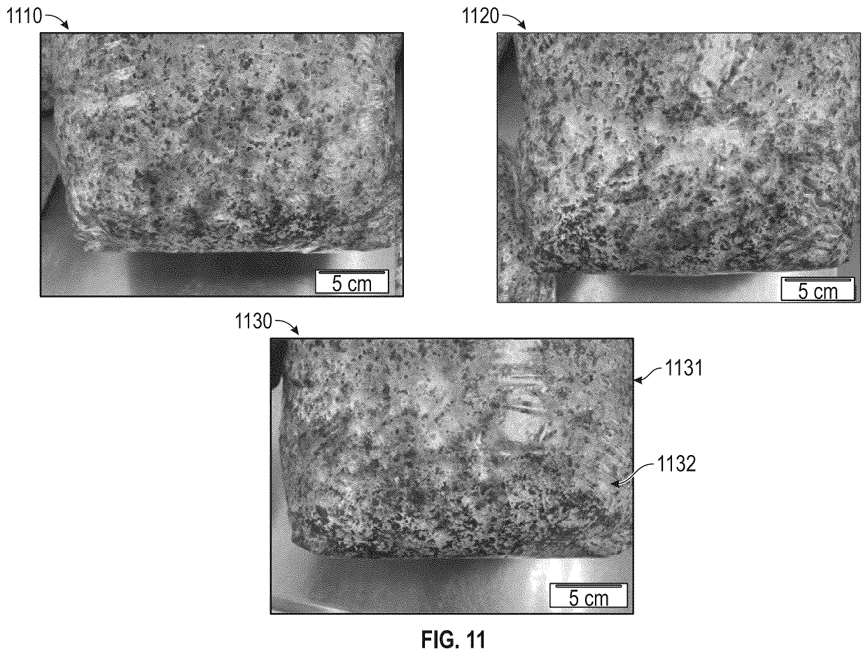

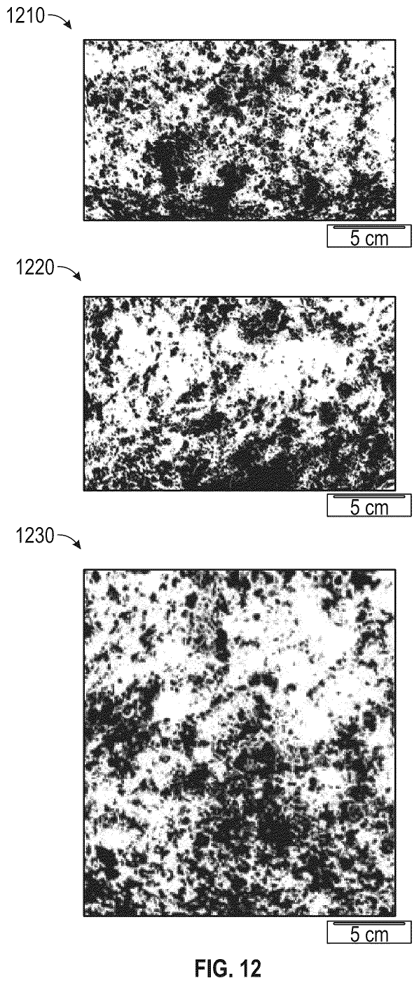

FIG. 9-12 illustrates visual colonization of pre-colonized substrate at 72-hours (FIGS. 9-10) and 96-hours (FIGS. 11-12) of incubation. FIGS. 10 and 12 show portions of the pre-colonized substrate depicted in the photographs of FIGS. 9 and 11 in black and white to emphasize visualization of mycelium biomass (in white). Images are shown to scale, with a scalebar of 5 cm included on each image.

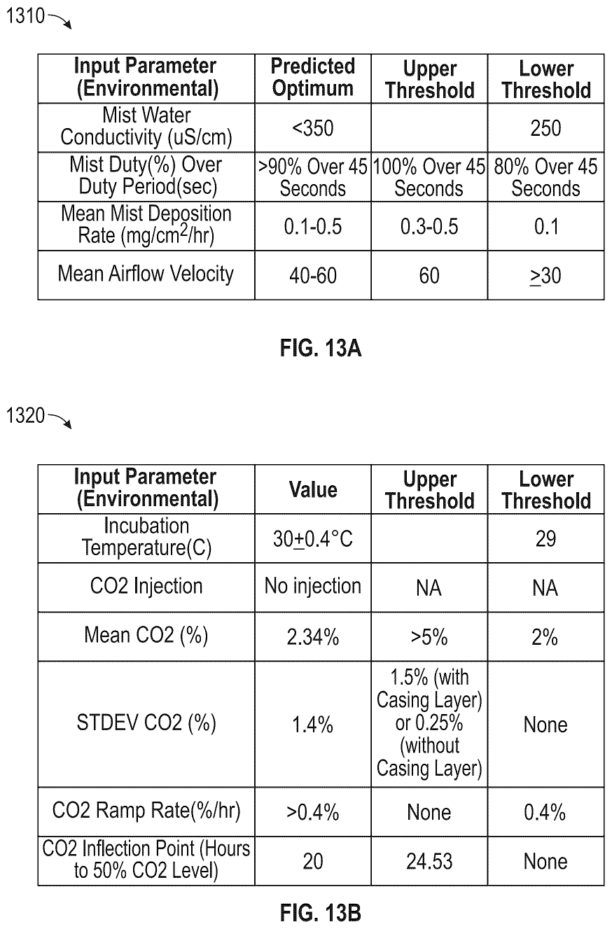

FIGS. 13A-B describe target and threshold values for environmental parameters. FIG. 13A tabulates data in 1310 describing target and threshold values for environmental mist and airflow parameters including the conductivity of the mist water, the periodicity of mist application (where the combination of duty fraction over a given duty period defines periodicity), mean mist deposition rate, and the mean airflow velocity over the whole aerial mycelium incubation period. In some embodiments, deviation from the described upper or lower thresholds may result in significant effects on aerial mycelium growth quality. FIG. 13B tabulates data in 1320 describing target and threshold values for environmental parameters including incubation temperature, whether CO₂ is actively injected into the incubation environment, the mean environmental CO₂ level over the whole aerial mycelium incubation period, the variance (standard deviation) of environmental CO₂ over the whole aerial mycelium incubation period, the ramp rate of CO₂ at the start of aerial mycelium incubation, and the inflection point (the hour at which 50% of the target mean CO₂ level is achieved). In some embodiments, deviation from the described upper or lower thresholds may result in significant effects on aerial mycelium growth quality. Statistical significance is indicated by a p-value as defined herein.

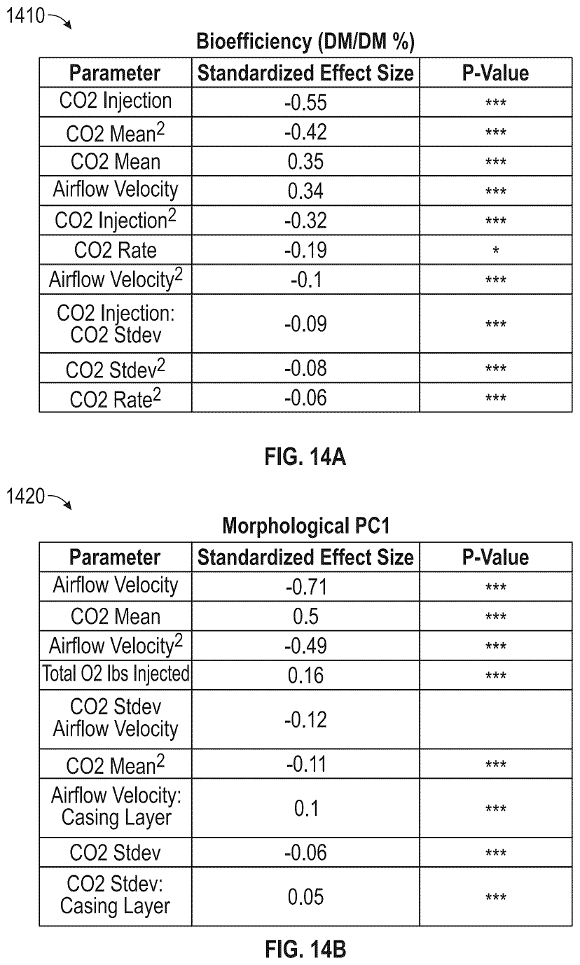

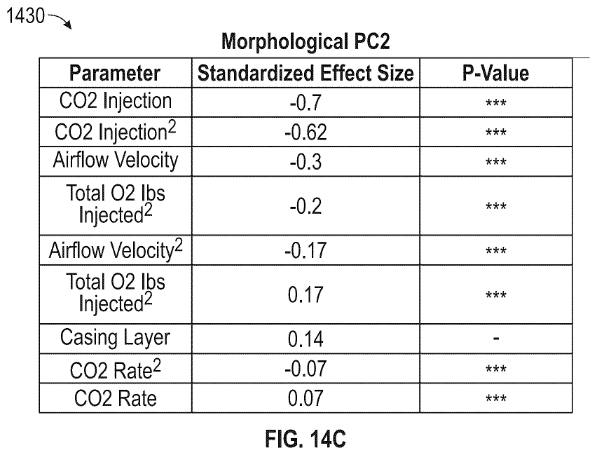

FIGS. 14A-C and 15A-C show ranked effects between growth qualities and environmental parameters, where increasing standardized effect size suggests greater sensitivity of the given growth quality to the given parameter. FIGS. 14A-C show gas exchange parameters 1410-1430 and FIGS. 15A-C show environmental parameters are depicted in 1510-1530. Morphological PC1 and PC2 describe morphological growth quality and bioefficiency describes the dry mass conversion efficiency of substrate to extra-particle aerial mycelium.

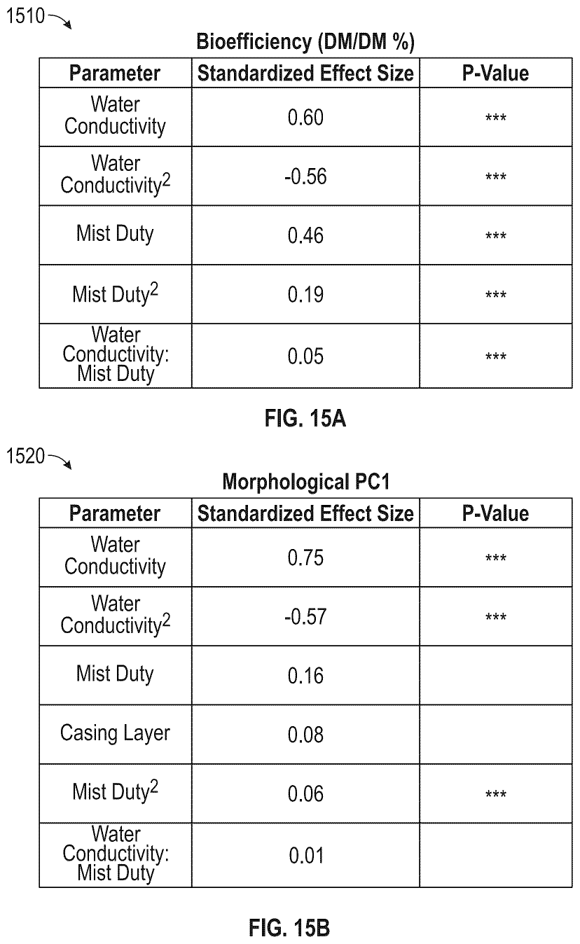

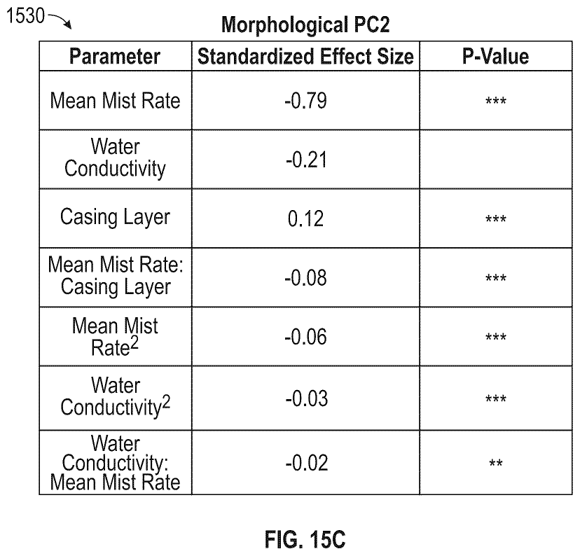

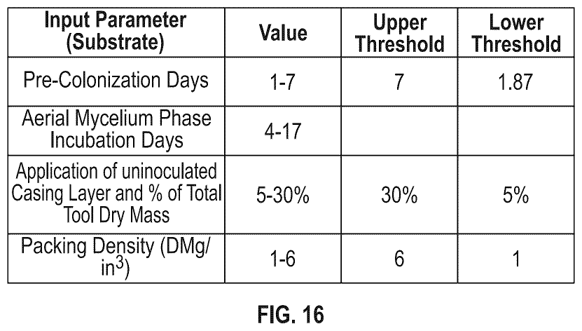

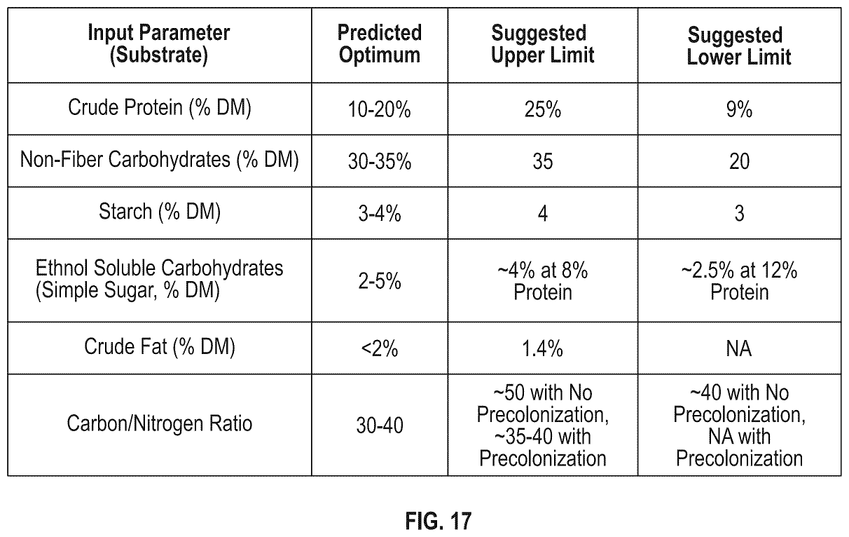

FIG. 16-17 describes target and threshold values for substrate incubation, casing layer, packing density, and composition parameters. In some embodiments, deviation from the described upper or lower thresholds may result in significant effects on aerial mycelium growth quality.

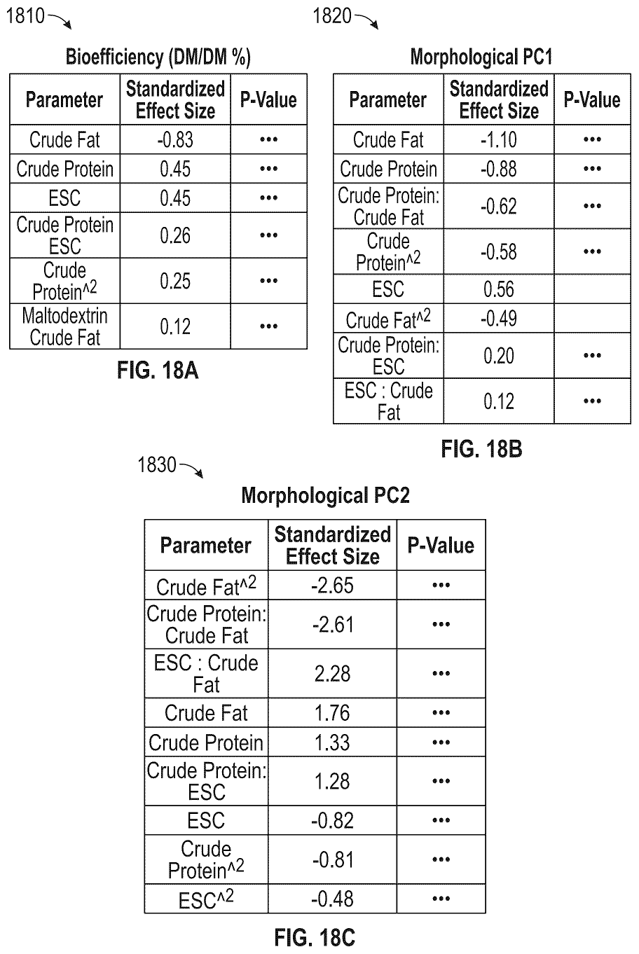

FIGS. 18A-C show ranked effects between defined substrate composition and growth qualities, where increasing standardized effect size suggests greater sensitivity of the given growth quality to the given parameter. Morphological PC1 and PC2 describe morphological growth quality and bioefficiency describes the dry mass conversion efficiency of substrate to mycelium.

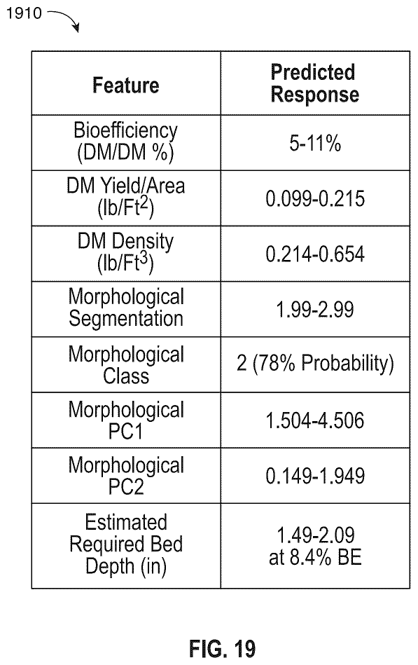

FIG. 19 describes the predicted aerial mycelium growth qualities 1910 resulting from operation of the aerial mycelium cultivation parameters described in any one of the above FIGS.

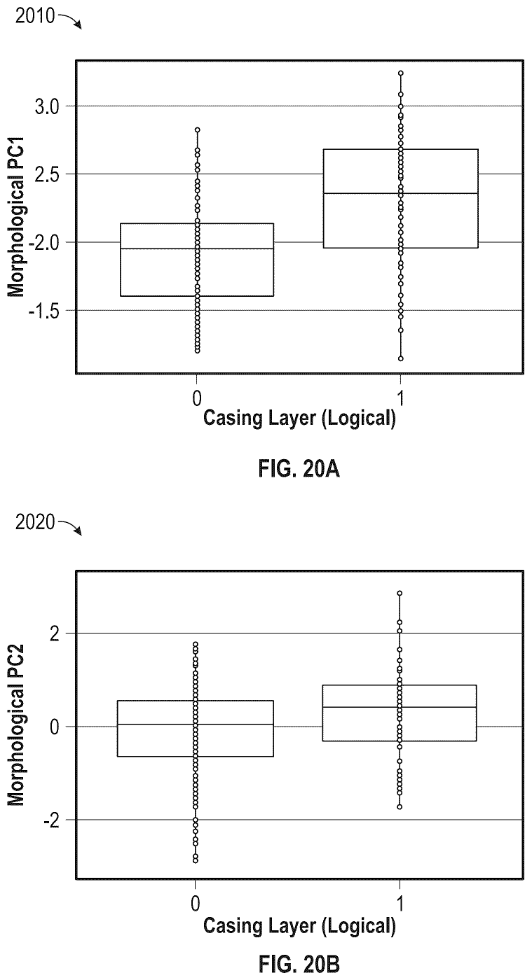

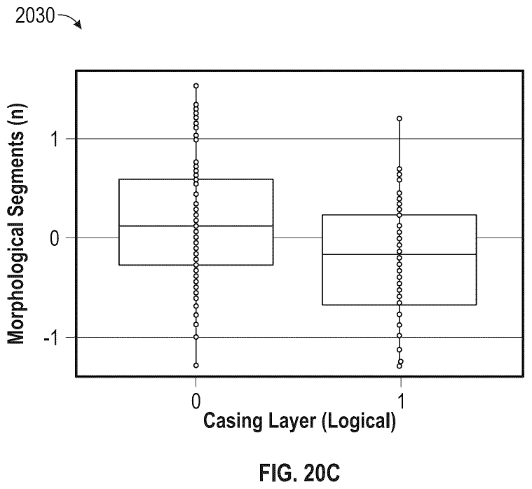

FIGS. 20A-C illustrate data showing morphological PC1 (2010), morphological PC2 (2020), and morphological segments (2030) comparing the full experimental population based on whether an uninoculated casing layer was or was not applied (i.e. “logical”), represented by a “1” and “0,” respectively. Morphological PC1 best represents growth height, volume, and degree of morphological segmentation, and morphological PC2 best represents general topological variance and directional bias, where increasing value in both PC1 and PC2 indicate greater homogeneity. Morphological segments indicate the number of discrete morphological features into which the sample can be segmented. Application of an uninoculated casing layer is associated with increased values in morphological PC1 and PC2 (p-val.<0.001 and p-val.=0.01, respectively, using a Kruskal-Wallace rank sum test), and reduced frequencies of discrete morphological segmentation (p-val.=0.001 per Kruskal-Wallace rank sum test). There were no significant effects on density or bioefficiency detected. The full response space suggests an overall significant positive effect on morphology independent of bioefficiency, total mass yield, or density.

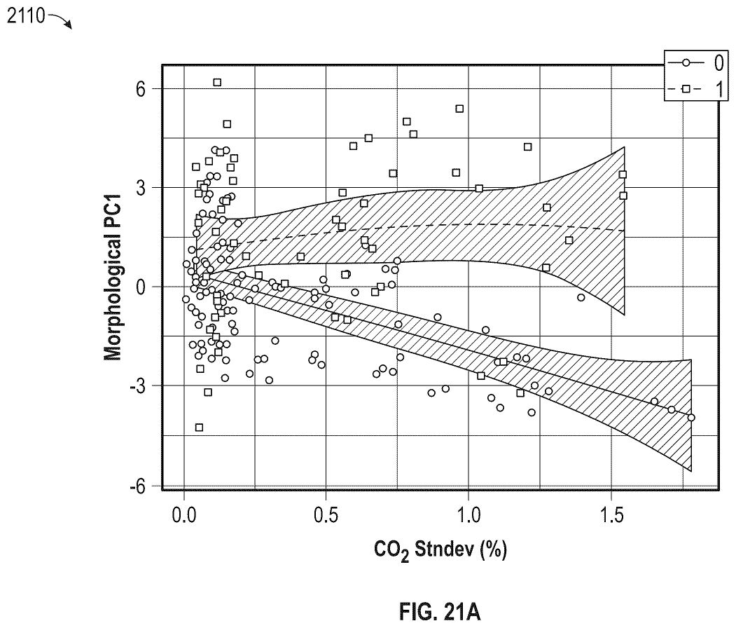

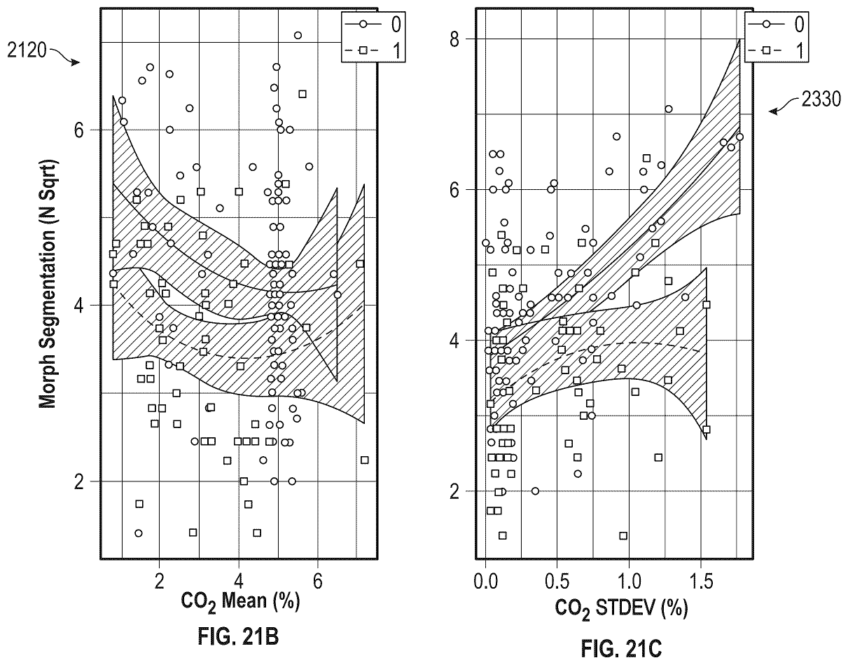

FIGS. 21A-C illustrate data with significant interaction between variance in environmental CO₂ and application of an uninoculated casing layer. 2110 shows morphological PC1 as a function of CO₂ variance, and 2120 and 2330 show morphological segmentation as a function of mean and variance in CO₂, respectively, coded by casing layer application (0=no casing layer, 1=with a casing layer). Shaded bands show a 95% confidence interval. There is significant interactivity with CO₂ variance, where the casing layer population shows a significant and qualitatively negative relationship between morphology where heterogeneity increases linearly with increasing CO₂ variance (p-val.<0.001 for morphological PC1 and p-val.=0.06 for morphological segmentation). There is also a meaningful reduction in morphological segmentation with casing layer application in the mean CO₂ range from about 2-4% (p-val.=0.03). Importantly, data indicate that the CO₂ parametrization is affected by whether a casing layer has been applied.

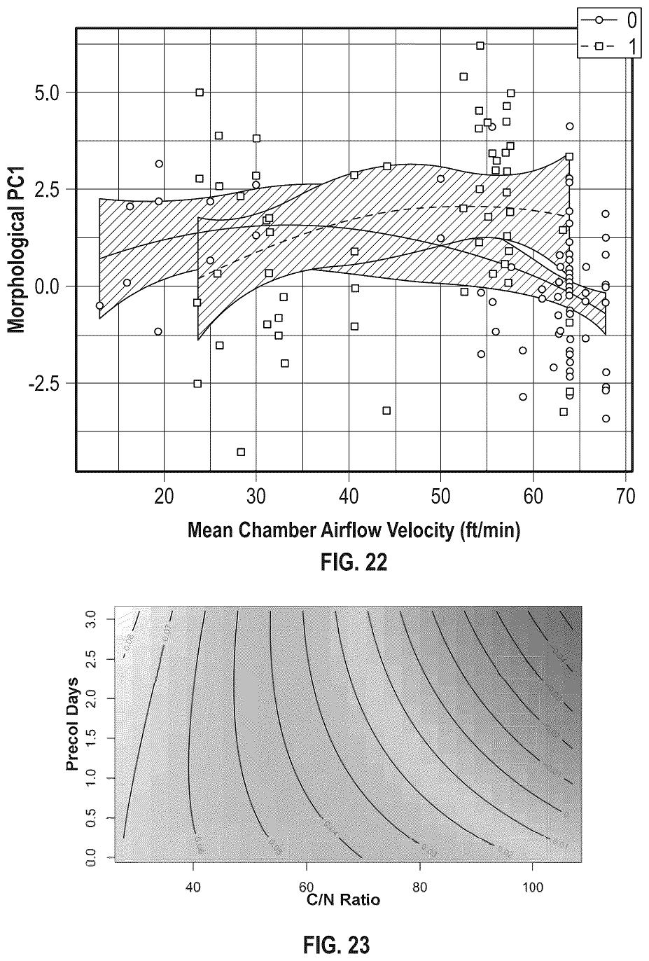

FIG. 22 illustrates the relationship between morphological PC1 and mean chamber airflow velocity. Surprisingly, there was a meaningful interactive relationship between airflow velocity and application of a casing layer (p-val.=0.001). Data show morphological PC1 as a function of mean airflow velocity, with data coded by casing layer application (1=with casing layer, 0=no casing layer). Data further show the highest values in morphological PC1 occurs at between 50-60 ft/min in combination with casing layer application. This region corresponds with the optimum, where a higher airflow velocity combined with a casing layer (1) mitigates the potential effect of higher airflow velocities occurring between 50-65 ft/min in the uncased population while (2) putting operational distance between the airflow velocity and lower velocities associated with occurrence of “islanding” (below about 20-30 ft/min), i.e., where islanding refers to aerial growth occurring as a series of discrete vertically oriented spires of mycelium with a lack of mycelium therebetween (a series of ‘islands’ rather than a contiguous panel).

FIG. 23 illustrates data showing a second-order fit of DM bioefficiency (where white is the highest value) as a combined function of precolonization and substrate C/N ratio. There was significant interactivity between pre-colonization days and C/N ratio (interactive at p-val.=0.03), where higher bioefficiency is achieved based on a combination of reduced C/N and longer pre-colonization time (3-days). Surprisingly, data suggest that the substrate composition presented here is dependent on pre-colonization.

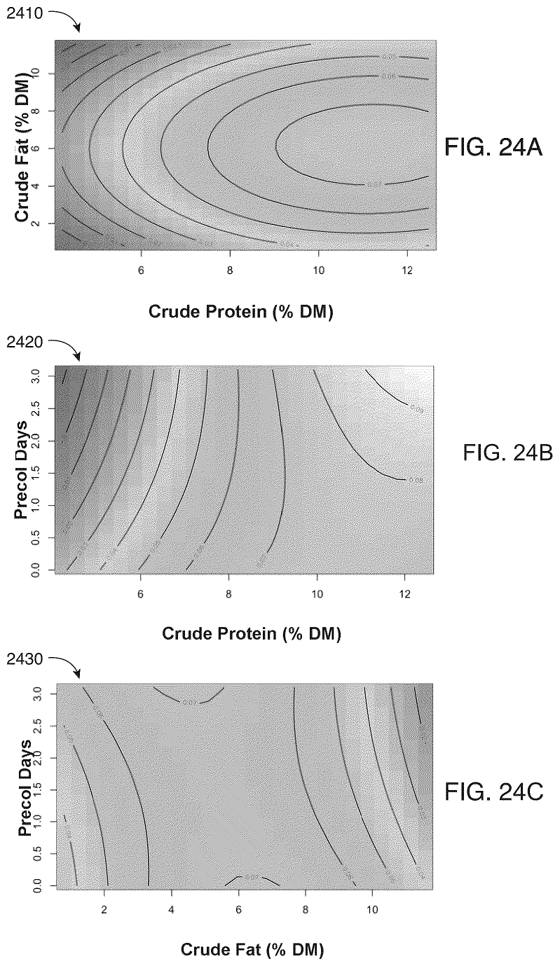

FIGS. 24A-C illustrate data demonstrating the effect of modifying the nutritional composition of growth medium. Defined nutritional composition was modified based on different weighting of raw substrate components, where some components heavily weight protein and others heavily weight both crude fat and protein. Data show the second-order relationship between DM bioefficiency (where white is the highest value) and crude protein content, crude fat content, and pre-colonization days (crude fat v. crude protein 2410, pre-colonization day v. crude protein 2420, and pre-colonization days v. crude fat 2430). There is significant interactivity between crude protein content and precolonization days (p-val.=0.008) and no interaction between crude fats and pre-colonization (p-val.=0.86). Data further show the interaction between protein loading and pre-colonization has a significant positive effect on bioefficiency (standard effect size=0.17) as well as protein loading alone (standard effect size=0.33), where fat has a significant second order effect where increasing values do not continue to return increasing bioefficiency (standard effect size=−0.33). These results surprisingly show interactivity between pre-colonization and protein content, where greater return on bioefficiency can be realized by leveraging this relationship.

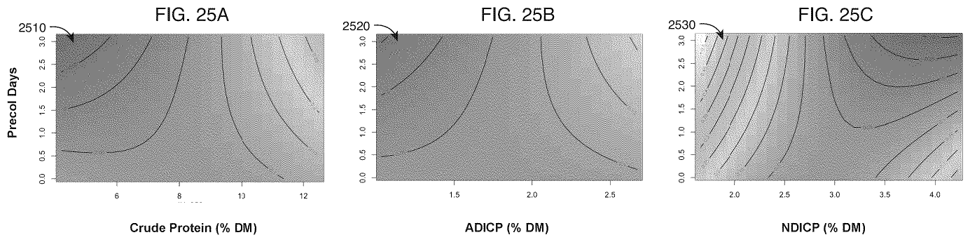

FIGS. 25A-C illustrate relationships between crude protein and pre-colonization. Relationships were evaluated between bioefficiency (where white is the highest value) and pre-colonization with different protein fractions: crude protein, with a significant positive interaction with pre-colonization as identified (p-val.=0.01) 2510; acid detergent insoluble crude protein (acid detergent fiber [cellulose and lignin] bound protein; ADICP), with a potentially meaningful positive interaction with pre-colonization (p-val.=0.09) 2520; and neutral detergent insoluble crude protein (protein bound to the neutral detergent fiber [encompassing most of the structural components of a plant]; NDICP), with a significant negative interactive effect with pre-colonization (p=.03) 2530. The soluble protein content demonstrated no meaningful relationships (not shown). Interactivity between total crude protein, bioefficiency, and pre-colonization best aligns with the ADICP fraction of the protein.

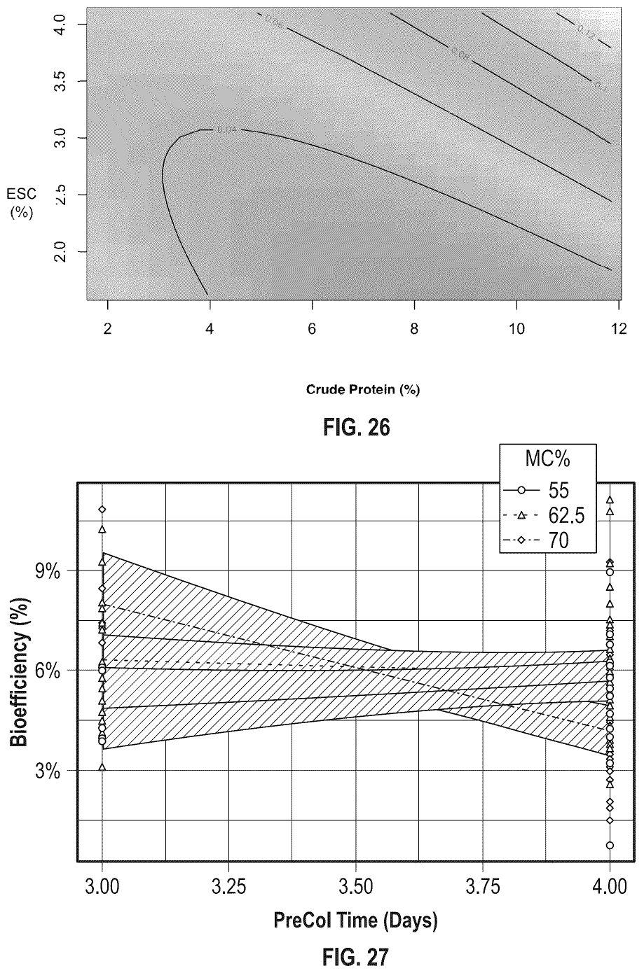

FIG. 26 illustrates data showing the relation of bioefficiency (where white is the highest value) as a combined function of crude protein and ethanol soluble carbohydrates (ESC).

FIG. 27 illustrates data for the relationship between bioefficiency and pre-colonization days. Data help explain the experimental rationale for chosen optima of moisture content of the substrate relative to the pre-colonization period, where optimal moisture content of the substrate is dependent on pre-colonization time.

### EXAMPLES

The following provides example compositions and cultivation methods for producing an extra-particle aerial mycelial growth, and ultimately an aerial mycelium. The examples are not intended to limit the embodiments described herein but are intended to illustrate how a growth medium composition and environmental conditions can be used to produce an aerial mycelium with certain material properties.

### Example 1

A pre-colonization phase growth medium was prepared with a nutrition profile comprising:
A protein content of at least 10% (w/w)
A non-fibrous carbohydrate content of less than 25% (w/w)
A total starch content of less than 4% by (w/w)
An ethanol-soluble carbohydrate content of less than 2.5% (w/w)
A fat content of less than 2% (w/w)
A carbon-to-nitrogen ratio at least 25 but less than 35.

The prepared dry growth medium was hydrated to a moisture content of at least about 60% to a maximum of about 70%. The hydrated pre-colonization phase growth medium was then sterilized for 1 hour at 15 psi in a pressure sterilizer.

After sterilization and cooling to less than 30° C. the pre-colonization phase growth medium was inoculated with millet grain spawn of Ganoderma sessile at a rate of about 15% (wet mass of spawn/bone dry mass of growth medium). The inoculated pre-colonization phase growth medium, or pre-colonization phase growth matrix, was placed into filter patch bags and heat sealed.

The pre-colonization growth matrix was incubated at room temperature for a pre-colonization period of three days in ambient light. After the pre-colonization period elapsed, the pre-colonization growth matrix was modified to create an aerial growth phase growth matrix with an aerial growth phase nutrition profile. The pre-colonization growth matrix was modified by grinding the pre-colonization growth matrix until it was reduced to discrete particles and mixing the ground pre-colonization growth matrix homogeneously with a simple sugar component.

The aerial growth phase nutrition profile comprising:
A protein content of at least 9% (w/w)
A non-fibrous carbohydrate content of at least 30% by (w/w)
A total starch content of less than 5% by (w/w)
An ethanol-soluble carbohydrate content of at least 2.5% (w/w)
A fat content of less than 2% (w/w)
A carbon-to-nitrogen ratio at least 25 but less than 40

The aerial growth phase growth matrix was loaded into a 4.5 cm tall and 810 ml volume tool at a packing density of 4.817 bone dry g/in³. The first 90% of the total mass loaded into the tray consisted of the aerial growth phase growth matrix, then the top 10% of the loaded mass consisted of an uninoculated, aerial growth phase growth medium with the aerial growth phase nutrition profile (i.e. a “casing” layer). The casing layer had a nominal thickness of 0.5 cm.

The loaded tool was placed into an environmentally controlled incubation chamber which excluded light, with the tool such that the casing layer growth medium interfaced with the environment in the incubation chamber. The environment in the chamber over a 7-day incubation period consisted of:
A temperature of 30C+/−a standard deviation of about 0.4.
A mean CO₂ level of at least about 2.345%.
A standard deviation of CO₂ level over the full 7-day incubation period of a maximum of less than 1.5.
A ramp rate from environmental CO₂ levels to the mean CO₂ level of at least about 0.4% per hour, reaching 50% of the mean CO₂ level before about 20 hours after incubation start.
A mean airflow velocity around the tray of a maximum of about 60 ft/min, and a minimum of about 40 ft/min.
A mean mist deposition rate of at least about 0.1 mg/cm²/hr, and a maximum of about 0.5 mg/cm²/hr.
Mist was introduced into the environment with an ultrasonic misting puck operating on at least a 90% duty cycle over a 45 second operation period (e.g., the mister pulses once for at least 40.5 seconds out of every 45 second period).
The mist water was comprised of tap water with a minimum conductivity of about 250 μS/cm.

### Example 2

A growth medium was prepared with a nutrition profile comprising:
At least 10.5% protein
A target total non-fiber carbohydrate content of about 31%.
A target total starch content of about 4.5%.
A minimum of about 4% ethanol soluble carbohydrates.
A maximum fat content of about 5%.
A maximum carbon/nitrogen ratio of about 45.

The prepared dry growth medium was hydrated to a moisture content of at least about 60% to a maximum of about 70%. The hydrated growth medium was then sterilized for 1 hour at 15 psi in a pressure sterilizer. Two filter patch bags were prepared.

After sterilization and cooling to less than 30° C. one of the prepared filter patch bags was inoculated with millet grain spawn of Ganoderma sessile at a rate of at least 35% (wet mass of spawn/bone dry mass of growth medium). This spawn was held for 13 days in refrigeration at 4° C. prior to use. The second bag was retained as uninoculated.

Growth medium was loaded into a 4.5 cm tall and 810 ml volume tool at a packing density of 4.817 bone dry g/in³. The first 80% of the total mass loaded into the tray consisted of the inoculated growth media, then the top 20% of the loaded mass consisted of the uninoculated growth media (i.e. a “casing” layer).

The loaded tool was placed into an environmentally controlled incubation chamber which excluded light, with the tool such that the casing layer growth media interfaced with the environment in the incubation chamber. The environment in the chamber over a 7-day incubation period consisted of:
A temperature of 30C+/−a standard deviation of about 0.4.
A mean CO₂ level of at least about 3.5%.
A standard deviation of CO₂ level over the full 7-day incubation period of a maximum of about 0.25%.
A ramp rate from environmental CO₂ levels to the mean CO₂ level of at least about 0.4% per hour, reaching 50% of the mean CO₂ level before about 20 hours after incubation start.
A mean airflow velocity around the tray of a maximum of about 60 ft/min, and a minimum of about 30 ft/min.
A mean mist deposition rate of at least about 0.1 mg/cm²/hr, and a maximum of about 0.6 mg/cm²/hr.
Mist was introduced into the environment with an ultrasonic misting puck operating on at least a 75% duty cycle over a 36 second operation period (e.g., the mister pulses one for 27.00-35.51 seconds out of every 36 second period).
The mist water was comprised of tap water with a minimum conductivity of about 100 μS/cm.

### Example 3

Using the protocol described above in Experiment 1, experiments were carried out to analyze the effect of moisture content of the pre-colonization phase growth medium and the length of the pre-colonization period. To this aim, a refined, more efficient growth process was developed, which aligns both pre-colonization phase growth medium moisture content and pre-colonization time, leading to a more efficient method of growing aerial mycelium.

As a result of the experimentation, an interactive relationship between pre-colonization phase growth medium moisture content and pre-colonization time was identified. Based on the relationship between pre-colonization phase growth medium moisture content and pre-colonization time, a manufacturing process was developed. For instance, where a 3-day pre-colonization time period is implemented, a higher moisture content of about 70% may be used. In another example, a pre-colonization time period may be 4 days or more, and the moisture content may be reduced to less than 65%. Each of these embodiments bioefficiency and yield were increased. The combination of a 63% moisture content and 4-day pre-colonization time period was confirmed to produce consistent, and highly bioefficient, aerial mycelium growth. The synergistic relationship between substrate moisture content and pre-colonization time periods are summarized in FIG. 27.

### Implementations

In some aspects, the present disclosure provides for an aerial mycelium, and for methods of making an aerial mycelium, wherein the aerial mycelium is a growth product of a fungus. In some embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces, Wolfiporia, Ceriporiopsis, Chlorociboria, Daedalea, Daedaleopsis, Daldinia, Ganoderma, Hypoxylon, Inonotus, Lenzites, Omphalotus, Oxyporus, Phanerochaete, Phellinus, Polyporellus, Porodaedalea, Pycnoporus, Scytalidium, Stereum, Trametes or Xylaria.

In some further embodiments, the fungus is a species of the genus Bondarzewia, Ceriporiopsis, Daedalea, Daedaleopsis, Fomitopsis, Ganoderma, Inonotus, Lenzites, Omphalotus, Oxyporus, Phellinus, Polyporellus, Polyporus, Porodaedalea, Pycnoporus, Stereum, Trametes or Xylaria. In some more particular embodiments, the fungus is selected from the group consisting of Bondarzewia berkeleyl, Daedalea quercina, Daedaleopsis spp., Daedaleopsis confragosa, Daedaleopsis septentrionalis, Fomitopsis spp., Fomitopsis cajanderi, Fomitopsis pinicola, Ganoderma spp., Ganoderma amboinense, Ganoderma applanatum, Ganoderma atrum, Ganoderma ibbose, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum, Ganoderma weberianum, Inonotus spp., Inonotus obliquus, Inonotus hispidus, Inonotus dryadeus, Inonotus tomentosus, Lenzites betulina, Phellinus spp., Phellinus igniarius, Phellinus gilvus, Polyporus spp., Polyporus squamosus, Polyporus badius, Polyporus umbellatus, Polyporus squamosus, Polyporus tuberaster, Polyporus arcularius, Polyporus alveolaris, Polyporus radicatus, Porodaedalea pini, Pycnoporus spp., Pycnoporus spp., Pycnoporus sanguineus, Pycnoporus cinnabarinus, Stereum spp., Stereum69rownia, Stereum hirsutum, Trametes spp., Trametes versicolor, Trametes elegans, Trametes suaveolens, Trametes ochracea, Trametes villosa, Trametes cubensis and Trametes pubescens.

In some other embodiments, the fungus is a pigment-producing fungus of a genus selected from the group consisting of Chlorociboria, Daldinia, Hypoxylon, Phanerochaete and Scytalidium.

In yet some other embodiments, the fungus is a species of the genus Ganoderma. In some further embodiments, the fungus is Ganoderma spp., Ganoderma amboinense, Ganoderma applanatum, Ganoderma atrum, Ganoderma australe, Ganoderma brownii, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum or Ganoderma weberianum.

### Food Implementation

In yet some other embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces or Wolfiporia.

In some further embodiments, the fungus is a species of the genus Pleurotus. In some more particular embodiments, the fungus is Pleurotus albidus, Pleurotus citrinopileatus, Pleurotus columbinus, Pleurotus cornucopiae, Pleurotus dryinus, Pleurotus djamor, Pleurotus eryngii, Pleurotus floridanus, Pleurotus nebrodensis, Pleurotus ostreatus, Pleurotus populinus, Pleurotus pulmonarius, Pleurotus sajor-caju, Pleurotus salmoneostramineus, Pleurotus salmonicolor or Pleurotus tuber-regium.

The various illustrative logics, logical blocks, modules, circuits and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general-purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage medium for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable medium includes both computer storage medium and communication medium including any medium that can be enabled to transfer a computer program from one place to another. A storage medium may be any available medium that may be accessed by a computer.

A software module may reside in random access memory (RAM), flash memory, read only memory (ROM), electrically programmable ROM (EPROM), electrically erasable programmable ROM (EEPROM), registers, hard disk, a removable disk, a CD ROM, or any other form of storage medium known in the art. A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blue ray disc where disks usually reproduce data magnetically, while discs reproduce data optically with lasers. Combinations of the above should also be included within the scope of computer readable medium. The processor and the storage medium may reside in an ASIC. The ASIC may reside in a user terminal. In the alternative, the processor and the storage medium may reside as discrete components in a user terminal.

Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein. Additionally, a person having ordinary skill in the art will readily appreciate that the terms “upper” and “lower” are sometimes used for ease of describing the figures and indicate relative positions corresponding to the orientation of the figure on a properly oriented page and may not reflect the proper orientation of a feature as implemented.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

The word “exemplary” is used herein to mean “serving as an example, instance, or illustration.” Any aspect or embodiment described herein as “exemplary” is not necessarily to be construed as preferred or advantageous over other aspects or embodiments. Various aspects of the novel systems, apparatuses, and methods are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of, or combined with, any other aspect described. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosures set forth herein. It should be understood that any aspect disclosed herein may be embodied by one or more elements of a claim.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Thus, as used herein, a phrase referring to “at least one of X, Y, and Z” is intended to cover: X, Y, Z, X and Y, X and Z, Y and Z, and X, Y and Z.

The headings provided herein, if any, are for convenience only and do not necessarily affect the scope or meaning of the devices and methods disclosed herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method of culturing an aerial mycelium, the method comprising:
preparing a mycelium growth medium with a nutrition profile comprising:
a protein content of at least 10% (w/w),
a non-fibrous carbohydrate content of at least 30% by (w/w),
a total starch content of less than 5% by (w/w),
an ethanol-soluble carbohydrate content of at least 4% (w/w),
a fat content of less than 5% (w/w), and
a carbon-to-nitrogen ratio at least 35 but less than 50;
hydrating the mycelium growth medium;
sterilizing the mycelium growth medium;
inoculating the mycelium growth medium with a fungal inoculum, thereby producing an inoculated mycelium growth medium; and
placing the inoculated mycelium growth medium into a growth environment configured to control a plurality of environmental conditions.

2. The method of claim 1, wherein the plurality of environmental conditions comprises: a temperature, a CO₂ content, an airflow velocity, a mist deposition rate, and a mist composition.

3. The method of claim 2, wherein the CO₂ content is at least 3.5% by volume of the growth environment.

4. The method of claim 3, wherein the CO₂ content varies 0.25% over an incubation time period.

5. The method of claim 3, wherein the CO₂ content increases during a first period of an incubation time period at a rate of at least 0.4% per hour to a target CO₂ level, and wherein the CO₂ content is maintained at the target CO₂ level during a second period of the incubation time period.

6. The method of claim 5, wherein the CO₂ content reaches 50% of the target CO₂ level within 20 hours of the beginning of the incubation time period.

7. The method of claim 2, wherein the airflow velocity is between 30 ft/min and 60 ft/min.

8. The method of claim 1, wherein the mycelium growth medium further comprises at least one simple sugar comprising at least arabinose, cellobiose, dextrin, dextrose, fructose, fucose, galactose, gentiobiose, glucosamine, glucose, lactose, lactulose, maltodextrin, maltose, maltotriose, mannose, melezitose, melibiose, sucrose, trehalose, xylose, or a combination thereof.

9. A method of culturing an aerial mycelium, the method comprising:
preparing a pre-colonization phase growth medium with a first nutrition profile;
inoculating the pre-colonization phase growth medium with a fungal inoculum to create a pre-colonization growth matrix;
incubating the pre-colonization growth matrix for a pre-colonization period;
modifying the first nutrition profile of the pre-colonization growth matrix to create an aerial growth phase growth matrix with a second nutrition profile;
applying a casing layer to the aerial growth phase growth matrix; and
placing the aerial growth phase growth matrix into a growth environment configured to control a plurality of environmental conditions.

10. The method of claim 9, wherein the first nutrition profile comprises:
a protein content of at least 10% (w/w);
a non-fibrous carbohydrate content of less than 25% (w/w);
a total starch content of less than 4% by (w/w);
an ethanol-soluble carbohydrate content of less than 2.5% (w/w);
a fat content of less than 2% (w/w); and
a carbon-to-nitrogen ratio at least 25 but less than 35.

11. The method of claim 9, wherein modifying the first nutrition profile comprises adding a simple sugar component to create the aerial growth phase growth matrix with the second nutrition profile.

12. The method of claim 10, wherein the second nutrition profile comprises:
a protein content of at least 9% (w/w);
a non-fibrous carbohydrate content of at least 30% by (w/w);
a total starch content of less than 5% by (w/w);
an ethanol-soluble carbohydrate content of at least 2.5% (w/w);
a fat content of less than 2% (w/w); and
a carbon-to-nitrogen ratio at least 25 but less than 40.

13. The method of claim 9, wherein the casing layer comprises an aerial growth phase growth medium comprising the second nutrition profile.

14. The method of claim 9, wherein the casing layer is between 10% (w/w) and 25% (w/w) of the aerial growth phase growth matrix.

15. The method of claim 9, wherein a thickness of the casing layer is between 0.25 cm and 2 cm.

16. The method of claim 9, wherein inoculating the pre-colonization phase growth medium comprises inoculating the pre-colonization phase growth medium at an inoculation rate.

17. The method of claim 16, wherein the inoculation rate is 15%, calculated as a spawn wet mass divided by an aerial growth phase growth medium dry mass.

18. The method of claim 9, wherein the plurality of environmental conditions comprises: a temperature, a CO₂ content, an airflow velocity, a mist deposition rate, a mist composition, and a mist duty cycle.

19. The method of claim 18, wherein the CO₂ content is at least 2.34% by volume of the growth environment.

20. The method of claim 19, wherein the CO₂ content varies less than 1.5% over an incubation time period.

21. The method of claim 19, wherein the CO₂ content increases during a first period of an incubation time period at a rate of at least 0.4% per hour to a CO₂ level, and wherein the CO₂ content is maintained at the CO₂ level during a second period of the incubation time period.

22. The method of claim 21, wherein the CO₂ content reaches 50% of the CO₂ level within twenty hours of beginning the incubation time period.

23. The method of claim 18, wherein the airflow velocity is between 40 ft/min and 60 ft/min.

24. The method of claim 18, wherein the mist deposition rate of between 0.1 mg/cm²/hour and 0.5 mg/cm²/hour.

25. The method of claim 18, wherein the mist composition comprises tap water with a minimum conductivity of at least 100 μS/cm.

26. The method of claim 18, wherein the mist duty cycle comprises a duty cycle of at least 75% to 100% and a duty cycle period of about 10 seconds to about 60 seconds.

27. The method of claim 9, wherein the pre-colonization phase growth medium further comprises a pre-colonization phase crude protein content, wherein the pre-colonization phase crude protein content is greater than 10% (w/w) and less than about 12% (w/w), and wherein a simple sugar component modifies the first nutrition profile such that an aerial growth phase non-fiber carbohydrate content is greater than 3% (w/w) but less than 3.5% (w/w).

28. The method of claim 27, wherein the pre-colonization phase crude protein content is greater than 8% (w/w) but less than 10% (w/w), and wherein the simple sugar component modifies the first nutrition profile such that the aerial growth phase non-fiber carbohydrate content is greater than 4% (w/w) but less than 4.5% (w/w).

29. The method of claim 9, further comprising a moisture content of at least 55% and less than 65%.

30. The method of claim 29, wherein the moisture content is about 63%.

31. The method of claim 9, wherein the pre-colonization period is four days or more.
